(19) United States
(12) Patent Application Publication
(10) Pub. No.: US 2019/0070166 A1
(43) Pub. Date: Mar. 7, 2019

(54) Optimized Method for Treating and Curing Arthritis, Diabetes, Multiple Sclerosis and Other Autoimmune Disease

(71) Applicant: Richard Postrel, Miami Beach, FL (US)

(72) Inventor: Richard Postrel, Miami Beach, FL (US)

(21) Appl. No.: 15/725,872

(22) Filed: Oct. 5, 2017

Related U.S. Application Data
(60) Provisional application No. 62/553,801, filed on Sep. 2, 2017.

(51) Int. Cl. A61K 31/4706; A61K 31/593; A61K 45/06

(52) U.S. Cl. CPC A61K 31/4706; A61K 45/06; A61K 31/593

(57) ABSTRACT

The present invention teaches a method, system and process for curing, treating and diagnosing arthritis, diabetes and other related autoimmune diseases. By intercepting and mitigating the disease process at the point of origination prevents the disease from developing and stops the lineage of cells causing disease symptoms. This fundamental system addresses cellular events where the immune reaction is emerging thereby preventing advance of the cascade that feeds the disease. Modulating activity of the errant protein at this initiating point in the immune response blocks the autoimmune cascade and prevents formation of secondary and tertiary effects that will characterize the disease. As the cascade progresses, the number of participating enzymes and pathways compounds so that each progression step further from the initiation point requires increasingly complex therapies. Thus, by treating the primary cause, secondary and tertiary symptoms do not appear. This avoids the side effects observed in multi-faceted approaches presently used to manage the disease symptoms rather than disease causation.

Autoimmune disorders afflict between an estimated one person in fourteen and one person in six in the US. One difficulty in getting accurate estimates is that autoimmune diseases have not always been recognized as such. As examples, diabetes type 1, rheumatoid arthritis, multiple sclerosis and Lupus were each recognized as diseases in their own rights long before the autoimmune etiology was laid out. Genetics are a risk factor as autoimmune disorders show increased prevalence in relatives of persons with autoimmune symptoms, even when they do not share living quarters. Immune reactive T-cells and antibody secreting B-cells can contribute to the autoimmune attacks.

The present invention results from a recognition of genetic factors underlying autoimmune attacks, in particular genetic factors relating to processes of destructive recycling of mitochondria in a process called mitophagy or autophagy and most particularly genetic factors relating to interactions involving transcription and expression of C-type lectin domain family 16 (CLEC16A) a gene that appears associated with various autoimmune diseases.

Accordingly, this invention teaches a method, system and process for curing, treating and diagnosing arthritis, diabetes and other related autoimmune diseases. This is accomplished by intercepting and mitigating the disease process at the point of origination thereby preventing the disease from developing and concomitantly checking the lineage of cells causing disease symptoms. This invention addresses cellular events where the immune reaction is emerging. The biologic cascade that provides the specialized cells feeding the disease is stopped at its inception, so formation of secondary and tertiary effects that will characterize the disease is not encountered. The need for drugs to treat these symptomatic events, with their inherent serious side effects, is thereby avoided.

The systems and methods of this invention by modulating errant protein activity at an initiating point in the immune response blocks the autoimmune cascade with its increasing number of involved metabolic pathways and associated transcription events and protein interactions. By treating the primary cause, secondary and tertiary sequelae do not follow. The early closure of the process at the inception stage avoids the spread of the early actions of the aberrant protein and its trailing pathway throughout multiple systems in multiple cell types. By treating the initiating events rather than its symptoms, the drugs with their side effects that are observed in multi-faceted treatment approaches presently being used to manage the relevant autoimmune disease symptoms are rendered redundant.

The range of targets of the immune system attacks is responsible for the many presentations of diseases with a common base. But even the common base of the immune system will have variations, in timing and intensity, sometimes locations of attack, associated with environmental factors such as stress, nutrition, food triggers, sleep cycles, exposure to antigenic substances (e.g., pollen).

Conventional treatments often address the symptoms with therapeutic pain killers including opioids, NSAIDS for pain—but with an anti-inflammatory (immune suppressant) effect, behavioral modification to reduce stress, physical therapy, immune suppressant corticosteroids such as prednisolone, hormone replacement such as insulin, etc.

Addison disease, Celiac disease—sprue (gluten-sensitive enteropathy), Dermatomyositis, Graves' disease, Hashimoto thyroiditis, Multiple sclerosis, Myasthenia gravis, Pernicious anemia, Reactive arthritis, Rheumatoid arthritis, Sjögren syndrome, Systemic lupus erythematosus and Type I diabetes are some of the more commonly occurring autoimmune diseases. According to the American Autoimmune Related diseases Association there are several score of recognized or suspected autoimmune diseases including: Addison's disease, Agammaglobulinemia, Alopecia areata, Amyloidosis, Ankylosing spondylitis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner ear disease (AIED), Autoimmune myocarditis, Autoimmune pancreatitis, Autoimmune retinopathy, Autoimmune urticaria, Axonal & neuronal neuropathy (AMAN), Bal disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss, Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hypogammalglobulinemia, IgA Nephropathy, IgG4-related sclerosing disease, Immune thrombocytopenic purpura (ITP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multiple sclerosis, Myasthenia gravis, Myositis, Narcolepsy, Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonnage-Turner syndrome Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjogren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (TTP), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo and Wegener's granulomatosis (or Granulomatosis with Polyangiitis (GPA)). These autoimmune disorders are characterized by self-directed immune activity targeting biomolecules of the host organism. Proteins—even proteins folded or bound in very precise and specific ways, nucleic acids, and cell fragments are examples of possible autoimmune targets. The varied nature of these and other biomolecules that can serve as autoantigens is a major source of the varied nature of symptoms.

Our immune system performs an amazingly complex function. It must recognize as “good” and therefore ignore all the healthy cells and tissues within our body (including our mutually beneficial microbiome) while—at the same time—it must attack and immobilize or destroy all alien “invaders,” foreign cells, viruses, bacteria, or fungi. Our immune system also recognizes simply “foreign” molecules of a size indicative of possible danger to our livelihoods. The awesomely complex immune system usually successfully protects our bodies while it identifies and eliminates billions of distinct infectious agents with which it comes in contact.

When it functions optimally, our immune system immediately recognizes an unwanted foreign virus or bacteria that has wandered into our body and initiates a spirited and robust attack on the invader before apparent illness. In suboptimal cases, after the invading microbe has reproduced multi-fold, our immune system will ideally step up intensity sufficient to allow us to recover from a cold after only a few days, but modulate the biocidal activity to minimize self-damage. When this precisely choreographed dance between the immune system and the tissues it is designed to protect goes awry we see symptoms of autoimmune diseases. In these diseases, the immune system has mistaken self for adversary with resultant attack on the very tissues we need for comfort and survival. Our immune system can be said to be mutineers attacking parts of the host body that the immune system cannot survive without.

We still do not fully understand what triggers autoimmune events or autoimmune disease. Sometimes it appears that an alien has presented a polypeptide fragment that is a close match for a polypeptide fragment in our self. Even so, most times the T-cells presenting such potential antigenic fragment will be eliminated and not clone to produce sufficient cells to mount an autoimmune disease attack. Scarlet fever is one example where the bacterium has a polypeptide fragment similar to one of our cardiac polypeptide sequences. One strain of virus used to prepare the 2009 version of tetravalent flu vaccine appears to have had a gene coding for a fragment of a protein expressed in our brain's reticular activating system. These instances are suspected sources of antibodies against our own tissues. Under normal healthy operations our body's immune system would have self-policed itself and eliminated T-cells and B-cells that have “mutinied”

Throughout human history, our exposure to constantly evolving infectious agents has required an evolutionary arms race. Our immune systems have evolved increasingly sophisticated countermeasures and recognition systems to counteract the increasing diversity of the infectious agents we come in contact with. While we have evolved, the “bugs” are also evolving, sometimes even coopting some of our evolved defenses for their reproductive purposes. Accordingly, this increasingly complex system comes with a cost of an increased chance of breaking down. We are always evolving as these bugs are right at the edge of the immune system's capacity.

And now over the last half century, our ability to make synthetic organic molecules as herbicides, insecticides, fertilizers and household products has been stressing our immune systems with new variances. These may be causing the immune system to increasingly make mistakes as it fine tunes with these novel types of substances. It is at the edge where the precise balance line becomes blurred, and our immune system attacks the host body with the result of unresolved autoimmune disease.

Possibly an underlying cause for this more frequent catastrophic autoimmune mistake in the immune system comes from the countless environmental “toxins” to which we are currently exposedtoxins that may mimic parts of our biomolecules such as hormones, thus interfering with the way the immune system communicates with the rest of the body.

Another line of hypotheses observes that since germ theory was accepted in the past century and a half and cleanliness and antibiotics have become more common, our immune systems have lost adversaries to fight and thus have had to seek out other targets. Whatever the reason(s), our bodies and our medical system are forced to acknowledge and address this increased activity of our immune systems against ourselves.

As an example of the complexities we need to account for in response to the increase in autoimmune attacks we need only look to our patent system where US Patent Application 20170051351, published Feb. 23, 2017, features: “A method for diagnosing and treating one or more autoimmune disorders (AID) in a patient, the method comprising: a) obtaining a biological sample from the patient; b) assaying nucleic acid from the sample to determine whether a genetic alteration in one or more of IL23R, LPHN2, PTPN22, TNFSF18, CRB1, IL10, TSSC1, IL18R1, ATG16L1, GPR35, DAG1, CYTL1, IL21, TNM3, PTGER4, ANKRD55, ERAP2, IL5, IL12B, 8q24.23, JAK2, LURAP1L, TNFSF15, FNBP1, CARD9, IL2RA, ANKRD30A, ZNF365, ZMIZ1, NKX2-3, INS, LRRK2, SUOX, EFNB2, SMAD3, SBK1, ATXN2L, ADCY7, NOD2, IKZF3, TYK2, FUT2, TNFRSF6B, PSMG1, CD40LG, and RBMX is present, wherein a genetic alteration in one or more of IL23R, LPHN2, PTPN22, TNFSF18, CRB1, IL10, TSSC1, IL18R1, ATG16L1, GPR35, DAG1, CYTL1, IL21, TNM3, PTGER4, ANKRD55, ERAP2, IL5, IL12B, 8q24.23, JAK2, LURAP1L, TNFSF15, FNBP1, CARD9, IL2RA, ANKRD30A, ZNF365, ZMIZ1, NKX2-3, INS, LRRK2, SUOX, EFNB2, SMAD3, SBK1, ATXN2L, ADCY7, NOD2, IKZF3, TYK2, FUT2, TNFRSF6B, PSMG1, CD40LG, and RBMX is correlated with presence of one or more AID in the patient; c) diagnosing the patient with one or more AID if a genetic alteration is present in one or more of IL23R, LPHN2, PTPN22, TNFSF18, CRB1, IL10, TSSC1, IL18R1, ATG16L1, GPR35, DAG1, CYTL1, IL21, TNM3, PTGER4, ANKRD55, ERAP2, IL5, IL12B, 8q24.23, JAK2, LURAP1L, TNFSF15, FNBP1, CARD9, IL2RA, ANKRD30A, ZNF365, ZMIZ1, NKX2-3, INS, LRRK2, SUOX, EFNB2, SMAD3, SBK1, ATXN2L, ADCY7, NOD2, IKZF3, TYK2, FUT2, TNFRSF6B, PSMG1, CD40LG, and RBMX; and d) administering an effective amount of one or more pharmaceutical agents listed in Tables 11 and 12 to the diagnosed patient.”

The 20170051351 application further teaches: “While a few studies have merged case genotypes from multiple diseases in a limited way, and a few loci have surfaced in independent GWAS studies across multiple autoimmune diseases, such as CLEC16A, first discovered in T1D, and subsequently in MS, RA, CD, PBC, JIA, and AA, the degree to which genetic variants associated with one disease may be associated with the risk of other autoimmune diseases has not been systematically examined. Clearly, having this information would provide new therapeutic avenues for the treatment of such disorders.”

The Clec16a locus has several SNP variants that have highlighted the importance of the gene and its expression for its involvement in several disease outcomes, including several associated with inflammation and autoimmune diseases. Clec16a interacts with neuregulin receptor degradation protein 1 (Nrdp1). Type 1 diabetes mellitus has been associated with Clec16a and deletion of Clec16a in mice increases Parkin expression. Clec16a regulates Nrdp1, its target Parkin and effectors porin, Mini, and Mfn2. Clec16a via Nrdp1 is understood to regulate autophagosomal trafficking during late mitophagy and pancreatic B-cell functions through control of its mitophagy operations.

Common variants in and around the CLEC16A gene locus have been found to be associated with type 1 diabetes. More recently, variants of this CLEC16A locus have been associated with a multiplicity of other inflammatory/autoimmune disorders, including, but not limited to: multiple sclerosis, inflammatory bowel disease and primary biliary cirrhosis. The CLEC16A gene product is a C-type lectin family member protein, having a biological function that has recently been reported to be linked at least in part to the mitophagy/autophagy pathway.

Specifically, a variant SNP rs12708716 has been associated with diabetes type 1. The SNP rs6498169 has been associated with higher risk of Multiple Sclerosis (MS). But protein expression is not uniform in all tissues throughout the body. Clec16A expression was not different in blood samples taken from MS patients compared with healthy controls. But in samples of isolated CD4 T cells homozygous for the rs12927355 risk allele (GG) both SOCS1 and CLEC16A proteins were expressed at elevated levels as compared to persons with the “non-risk” allele (AG/AA). Since MS is not present at birth, it is obvious that not all carriers of the “risk” gene will present with MS. While the rs12927355 allele is associated with MS in a significantly statistical fashion, there may be other SNPs or variants that associate with MS or other autoimmune diseases. Clearly another variant, rs12708716 also is associated with auto-immune activity, i.e., at least, diabetes type 1.

While it is possible to screen for aberrant levels of protein expression as an indicator or autoimmune risk or confirmation, this is not a preferred assay. Preferably candidate persons who would benefit from the instant invention would be assessed for auto-immune symptoms and/or variation(s) in their CLEC16A locus, in a coding or non-coding region. Other non-preferred assays would involve monitoring expression of mitophagic pathway proteins, levels of metabolism (heat of mitochondrial activity), quality and quantity of the mitophagic associated RNAs, PINK1 phosphorylation, ROS levels, ubiquitination, deubiquitination, and/or fusion/fission balance of mitochondria. Erythrocyte sedimentation rate and/or C reactive protein tests are inexpensive and quick methods for confirming immune system involvement. A preferred assay would involve reading or assessing (e.g., by chip analysis) one or more nucleic acid sequences associated with the CLEC16A locus.

Mitophagy is a term used in reference to the cell's destructive recycling of mitochondrial structure and biochemicals. The process is aptly described in Wikipedia as:
Mitophagy is the selective degradation of mitochondria by autophagy. It often occurs to defective mitochondria following damage or stress. The term was coined by J. J. Lemasters in 2005. [Lemasters, J (2005). “Selective mitochondrial autophagy, or mitophagy, as a targeted defense against oxidative stress, mitochondrial dysfunction, and aging”. Rejuvenation Research. 8:3-5. doi:10.1089/rej.2005.8.3] Mitochondrial fragments had been seen in liver lysosomes as early as 1962, [Ashford, T P; Porter, K R (1962). “Cytoplasmic components of hepatic cell lysosomes”. The Journal of Cell Biology. 12:198-202. PMC 2106008 Freely accessible. PMID 13862833. doi:10.1083/jcb.12.1.198] and a 1977 report suggested that “mitochondria develop functional alterations which would activate autophagy”. [Beaulaton, J; Lockshin, KR (1977). “Ultrastructural study of the normal degeneration of the intersegmental muscles of Anthereae polyphemus and Manduca sexta (Insecta, Lepidoptera) with particular reference of cellular autophagy”. [Journal of Morphology. 154: 39-57. doi:10.1002/jmor.1051540104].
Mitophagy is key in keeping the cell healthy. It promotes turnover of mitochondria and prevents accumulation of dysfunctional mitochondria which can lead to cellular degeneration. It is mediated by Atg32 (in yeast) and NIX and its regulator BNIP3 in mammals. Mitophagy is regulated by PINK1 and parkin proteins. In addition to the selective removal of damaged mitochondria, mitophagy is also required to adjust mitochondrial numbers to changing cellular metabolic needs, for steady-state mitochondrial turnover, and during certain cellular developmental stages, such as during cellular differentiation of red blood cells. [Youle, R; Narendra, D (2011). “Mechanisms of mitophagy”. Nature Reviews Molecular Cell Biology. 12: 9-14. doi:10.1038/nrm3028].

The present invention features novel systems and methods for arresting progression of mitophagic/autophagic associated autoimmune diseases. The invention includes methods of treatment using one or more compounds administered for their effects on mitophagy/autophagy.

Autophagy is the general term describing a cell's orchestrated homeostatic process to eliminate unwanted proteins and damaged or redundant organelles such as mitochondria. Autophagy is a controlled process involving many cellular elements that promote and many cellular elements that slow or arrest the programmed degradative processes. When the autophagic process is applied to mitochondria the process is often called mitophagy. The autophagic process is also applied for removing intracellular microbial pathogens. Leading up to and during the autophagic process, cell signaling pathways sense different types of cell activities or stresses, including, e.g., nutrient shortfall and/or microbial invasion and vary responses depending on the sensed inputs.

Mitophagy is necessary to discard and recycle excess or dysfunctional mitochondria to maintain balanced cellular respiration. Many enzymes are involves in the process, some which encourage reprocessing and some which maintain the status quo. High-throughput expression microarray and chromatin occupancy analyses reveal that Pdx1 regulates the expression of Clec16a, a type 1 diabetes gene which is itself a key mediator of mitophagy through regulation of the E3 ubiquitin ligase Nrdp1. Reduction of Pdx1 levels impairs the fusion of autophagosomes containing the targeted mitochondria to lysosomes for degradative disassembly during mitophagy.

Mitochondrial stress or one or a combination of several other signals can initiate the mitophagy process. The stress may be from any of a variety of sources including, but not limited to: a chemical stress, lack of nutrition, DNA mutation, ROS, damaged mitochondrial membrane proteins, misfolded proteins, compromised membrane integrity, decreased mitochondrial membrane potential, etc. Multiple stresses may co-present, for example, ROS acting on the membrane and/or membrane proteins can compromise the membrane potential and cause protein and DNA damages. PINK1 degradation may be slowed causing it to accumulate on the damaged mitochondrion. Pink1 then will autoactivate (phosphorylate) to phosphorylate and activate mitochondrial ubiquitin, both mono and poly species including both Lys 48 and Lys63 on the poly chain. This activates Parkin, elicits mitochondrial migration and proteasomal degradation.

Multiple enzymes, especially in the ubiquitination/deubiquitination pathways, can participate in controlling the process. These include, but are not limited to: E2 enzymes, UBE2N, UBE2L3, UBE2D2/3, Parkin E3, USP30, USP15, UBR1, UBR2, UBR4, (ATPIF1, TOMM7, PARL, SIAH3, SREBF1 with special respect to PINK1), (HSPA1L, Hsp72, HK1, and HK2 important for Parkin translocation), (BAG4 and USP8 acting as Parkin translocation suppressors), HK2 regulated by AKT, p62/SCISTM1 regulated by TBK1, optineurin, PI3K activator Ambral, NBR1, etc. Accordingly, there are multiple opportunities for modulating mitophagic activity. The present invention focuses on CLEC16A.

Expression of the variant CLEC16A genes has varied effects depending on the variant, the cell type and other genes and conditions within the cell. The CLEC16A protein is only one of several thousands of different proteins in a cell. Many factors are involved in initiating and continuing autophagic/mitophagic activity. The different cell types express different proteins and amounts of proteins to satisfy their metabolic needs. CLEC16A variants are associated with different expression profiles for several other cell proteins. The CLEC16A protein is only one of several thousands of different proteins in a cell. These variables help explain how a variant of one gene or protein can present with different manifestations. However, certain variants appear to follow a pattern in their related autoimmune presentations.

Stresses in general, for example lack of one or more nutrient or vitamin, tip a cell's metabolism in support of autophagy. A variant CLEC16A would be expected to have a different response to one or more forms of stress even in the same cell, so when looking at different cells the variance is even more pronounced. But at the base, the mitophagic/autophagic processes mediated in part by the CLEC16A protein can be modulated in favor of arresting inflammation in several cells types and autoimmune diseases presenting in many others.

An important stage for inducing autophagy where several branches that modulate autophagy come together is membrane nucleation which is controlled by the ULK complex and Beclin1. Inhibitors of positive regulators of the ULK complex and Beclin1 have been demonstrated to slow or arrest autophagy. Such inhibitors include, but are not limited to: MAP kinases, JNK1, ERK and p38. Another requirement involves induction of Atg protein and LC3 proteins for vesicle expansion and formation.

Insulin and some growth factors induce or stimulate phosphatidylinositol 3 kinase early in the autophagic process. Inhibitors of the class III PI3 kinases can block autophagy. In a later step of the autophagic process, inhibitors that inhibit lysosome acidification essentially block the formation of autophagosome and autophagic degradation and thereby arrest the degradative processes.

The phosphoinositide-3 kinase (PI3K) pathway has been proposed as an important target in breast cancer treatment. PI3Ks are members of a family of lipid kinases that phosphorylate the 3-hydroxyl group of phosphoinositides. Activation of PI3Ks is normally initiated by a growth factor or other ligand that binds to its cognate receptor tyrosine kinase (RTK). These RTKs include, but are not limited to: the human epidermal growth factor receptor (HER) family, the insulin receptor and the insulin-like growth factor 1 receptor (IGF-1R). This binding removes the inhibitory effect with result of full activation of PI3K. This activated kinase system then catalyzes phosphorylation of phosphatidylinositol bisphosphate (PIP2) to phosphatidylinositol triphosphate (PIP3). PIP3 then acts as a docking site for Akt, a serine/threonine kinase that is the central mediator of the PI3K pathway, and phosphoinositide-dependent kinase 1 (Pdk1). Once localized at the cell plasma membrane, Akt is phosphorylated and stimulates protein synthesis and cell growth by activating mammalian target of rapamycin (mTOR) through effects on the intermediary tuberous sclerosis 1/2 complex. Akt is a throttle point for TSC1/2 inhibition of the mTOR raptor induced protein synthesis and cell growth; p53, BAD, FOXO/FasL,Bim to remove their brake on apoptosis and inhibit GSK3 to slow its stimulation of metabolism.

The PI3K pathway is thus integral to diverse cellular functions, including, but not limited to: cellular metabolism and proliferation, differentiation, and survival. Several compounds originally applied in human disease context for treating breast cancer are viable candidates for reformulating as a management or treatment tool to arrest progression of CLEC16A associated autoimmune diseases. Recent evidence suggests a role of CLEC16A in asthma. The present invention's focus on CLEC16A mediated processes thus is applicable to many autoimmune diseases as a group and also to some inflammatory reactions of the immune system.

Vps34 acts to phosphorylate PIP to form PI3P at the pre-autophagosome or endosome thus recruiting one or more FYVE and PX domain containing proteins. Vps34 also associates with protein kinase Vps15 in different protein complexes, and here participates in membrane trafficking and protein sorting pathways. Because of the location, PI3P produced by Vps34 is essential for autophagosome and phagosome maturation as well as for NOX2 mediated Reactive Oxygen Species (ROS) production. Thus VPS34 is another enzyme playing a key role in autophagy, as well as pathogen uptake and the killing by innate immune cells.

The process of autophagy is an important contributor to normal cell function for removal and recycling of fragmented or misfolded proteins and of damaged organelles. Autophagy has been put to another use in the immune system in that autophagy is the process that mediates intracellular Toll Like Receptor (TLR) activation by bringing cytoplasmic antigens in contact with TLR in the lysosome where it promotes cross-presentation of intracellular antigens on MHCII. T cell-specific loss of Vps34 has been observed to impair invariant NKT cell development and peripheral T cell homeostasis. This ultimately causes intestinal inflammation and wasting syndrome.

With respect to modulating the autophagic system N4-(7-Chloro-4-quinolinyl)-N1,N1-dimethyl-1,4-pentanediamine (chloroquine) may be a strong candidate. It is often administered as a diphosphate salt in some cancer treatments and may be the most widely tested and accepted with respect to human use in breast cancer treatment in this realm. Chloroquine accumulates inside the acidic compartments of the cell, including endosomes and lysosomes. This accumulation leads to inhibition of lysosomal enzymes that require an acidic pH, and prevents fusion of endosomes and lysosomes. It is a lysosomotropic agent that prevents endosomal acidification. Chloroquine inhibits autophagy as it raises the lysosomal pH, which leads to inhibition of both fusion of autophagosome with lysosome and lysosomal protein degradation. US Patent Application 20050080113 suggests that chloroquine may be used with “an agent for the prophylaxis or treatment of pain and/or suppression of activation and/or inhibition of formation of osteoclast, which contains a p38 MAP kinase inhibitor and/or a TNF-α production inhibitor” thus providing another example, that a pharmaceutical composition that includes chloroquine as a major active ingredient would very likely be safe and acceptable for human use.

While chloroquine is generally considered as safe for human use, one possible side effect may involve reduced glutathione in some cells. Accordingly, several embodiments may include GSH and/or other antioxidant supporting components. Compounds such as N-acetylcysteine (NAC), α-lipoic acid (ALA), S-adenosylmethionine (SAMe), colecalciferol (vitamin D3), mustard seed or other Se rich phyto-supplements, etc. can be co-administered with any bioactive to support and maintain glutathione levels. Divalent cations and chelates, preferably predominant in Mg⁺⁺ and Ca⁺⁺ can be used to support enzymatic activities and membrane integrity and transport from possible stress associated with the chief bioactive component(s), but potentially including some multivalent ions, e.g., transition metals such as copper or iron may be incorporated into the formulation for stability, bioavailability, cofactor or nutritional support, etc.

Additional inhibitors relating to enzymes in this autophagy pathway include, but are not limited to: 2H-dibenzo[cd,g]indazol-6-one (SP600125), 1,4-Diamino-2,3-dicyano-1,4-bis(2-aminophenylthio)-butadiene (U0106), Bafilomycin A1 (C₃₅H₅₈O₉), (1S,6bR,9aS,11R,11bR)-11-(acetyloxy)-1,6b,7,8,9a,10,11,11b-octahydro-1-(methoxymethyl)-9a,11b-dimethyl-3H-Furo[4,3,2-de]indeno[4,5-h]-2-benzopyran-3,6,9-trione (Wortmannin), 4-(4-(4-fluorophenyl)-2-(4-(methylsulfinyl)phenyl)-1H-imidazol-5-yl)pyridine (5B203580), 4-(4-Fluorophenyl)-2-(4-hydroxyphenyl)-5-(4-pyridyl)-1H-imidazole (5B202190), 2-(4-morpholino)-8-phenyl-4H-1-benzopyran-4-one (LY294002), 3-methyladenine (3-MA), 2-Methyl-2-{4-[3-methyl-2-oxo-8-(quinolin-3-yl)-2,3-dihydro-1H-imidazo[4,5-c]quinolin-1-yl]phenyl}propanenitrile (Dactolisib), 2-(1H-indazol-4-yl)-6-((4-(methylsulfonyl)piperazin-1-yl)methyl)-4-morpholinothieno[3,2-d]pyrimidine (Pictilisib), (S)-2-(1-(9H-purin-6-ylamino)propyl)-5-fluoro-3-phenylquinazolin-4(3H)-one (Idelalisib), (S)-2-(1-(9H-purin-6-ylamino)propyl)-5-fluoro-3-phenylquinazolin-4(3H)-one (AZD8186), (R)-8-(1-(3,5-difluorophenylamino)ethyl)-N,N-dimethyl-2-morpholino-4-oxo-4H-chromene-6-carboxamide (AZD8186), L-serine, L-arginyl-L-glutaminyl-L-isoleucyl-L-lysyl-L-isoleucyl-L-tryptophyl-L-phenylalanyl-L-glutaminyl-L-asparaginyl-L-arginyl-L-arginyl-L-methionyl-L-lysyl-L-tryptophyl-L-lysyl-L-lysyl-L- seryl-L-aspartylglycylglycyl-O-phosphono-L-tyrosyl-L-methionyl-L-aspartyl-L-methionyl-(740Y-P), 4-(4-cyano-2-fluorophenyl)-2-morpholino-5-(2H-1,2,4-triazol-3-yl)thiophene-3-carbonitrile (PF-4989216), N-(3-{[(3-{[2-chloro-5-(methoxy)phenyl]amino}quinoxalin-2-yl)amino]sulfonyl}phenyl)-2-methylalaninamide (Pilaralisib), 6-((4-(cyclopropylmethyl)piperazin-1-yl)methyl)-2-(5-fluoro-1H-indol-4-yl)-4-morpholinothieno[3,2-d]pyrimidine (PI-3065), 4′-(cyclopropylmethyl)-N2-4-pyridinyl-[4,5′-Bipyrimidine]-2,2′-diamine (PIK-III), 1-[[2-[(2-chloro-4-pyridinyl)amino]-4′-(cyclopropylmethyl)[4,5′-bipyrimidin]-2′-yl]amino]-2-methyl-2-Propanol (VPS34-IN1), 5-(6-(3-methoxyoxetan-3-yl)-7-methyl-4-morpholinothieno[3,2-d]pyrimidin-2-yl)pyrimidin-2-amine (GNE-317), 2-amino-8-ethyl-4-methyl-6-(1H-pyrazol-5-yl)pyrido[2,3-d]pyrimidin-7-one (Voxtalisib), 2-(3,4-dihydroxyphenyl)-3,5,7-trihydroxy-4H-1-Benzopyran-4-one (Quercetin), Ethyl 6-(5-(phenylsulfonamido)pyridin-3-yl)imidazo[1,2-a]pyridine-3-carboxylate (HS-173), N-[5-[4-[5-[[(2R,6S)-2,6-dimethyl-4-morpholinyl]methyl]-2-oxazolyl]-1H-indazol-6-yl]-2-methoxy-3-pyridinyl]-methanesulfonamide (GSK2292767), N-((S)-1-(7-fluoro-2-(pyridin-2-yl)quinolin-3-yl)ethyl)-9H-purin-6-amine (AMG319), 5-(2-amino-8-fluoro[1,2,4]triazolo[1,5-a]pyridin-6-yl)-N-(1,1-dimethylethyl)-3-pyridinesulfonamide (CZC24832), α,α-dimethyl-4-[5,6-dihydro-2-[3-methyl-1-(1-methylethyl)-1H-1,2,4-triazol-5-yl] imidazo[1,2-d][1,4]benzoxazepin-9-yl]-1H-pyrazole-1-acetamide (Taselisib), 5-[8-methyl-9-(1-methylethyl)-2-(4-morpholinyl)-9H-purin-6-yl]-2-pyrimidinamine (VS-5584), 3-(2,4-diamino-6-pteridinyl)-phenol (TG100713), 1-(4-(3-ethyl-7-morpholino-3H-[1,2,3]triazolo[4,5-d]pyrimidin-5-yl)phenyl)-3-(4-(1-methylpiperazine-4-carbonyl)phenyl)urea (PKI-402), 2-((4-amino-1H-pyrazolo[3,4-d]pyrimidin-1-yl)methyl)-5-methyl-3-o-tolylquinazolin-4(3H)-one (PIK-293), (E)-5-((5-(4-fluorophenyl)furan-2-yl)methylene)thiazolidine-2,4-dione (CAY10505), 5-(7-(methylsulfonyl)-2-morpholino-6,7-dihydro-5H-pyrrolo[2,3-d]pyrimidin-4-yl)pyrimidin-2-amine (CH5132799), 6-amino-N-[3-[4-(4-morpholinyl)pyrido[3′,2′:4,5]furo[3,2-d]pyrimidin-2-yl]phenyl]-3-Pyridinecarboxamide (YM201636), 2-amino-N-[2,3-dihydro-7-methoxy-8-[3-(4-morpholinyl)propoxy]imidazo[1,2-c]quinazolin-5-yl]-5-Pyrimidinecarboxamide, (Copanlisib), (Z)-5-((2,2-difluorobenzo[d][1,3]dioxol-5-yl)methylene)thiazolidine-2,4-dione (AS-604850), 2-((4-amino-3-(3-hydroxyphenyl)-1H-pyrazolo[3,4-d]pyrimidin-1-yl)methyl)-5-methyl-3-otolylquinazolin-4(3H)-one (PIK-294), N-hydroxy-2-(((2-(6-methoxypyridin-3-yl)-4-morpholinothieno[3,2-d]pyrimidin-6-yl)methyl)(methyl)amino)pyrimidine-5-carboxamide (CUDC-907), 2-methyl-1-[[2-methyl-3-(trifluoromethyl)phenyl]methyl]-6-(4-morpholinyl)-1H-Benzimidazole-4-carboxylic acid (GSK2636771), 8-(6-methoxypyridin-3-yl)-3-methyl-1-(4-(piperazin-1-yl)-3-(trifluoromethyl)phenyl)-1Himidazo[4,5-c]quinolin-2(3H)-one maleic acid (BGT226), (Z)-5-((5-(4-fluoro-2-hydroxyphenyl)furan-2-yl)methylene)thiazolidine-2,4-dione (AS-252424), 6,7-Bis(3-hydroxyphenyl)pteridine-2,4-diamine (TG100-115), 8-chloro-2-phenyl-3-[(1S)-1-(9H-purin-6-ylamino)ethyl]-1(2H)-Isoquinolinone (Duvelisib), 1-(4-(4-(dimethylamino)piperidine-1-carbonyl)phenyl)-3-(4-(4,6-dimorpholino-1,3,5-triazin-2-yl)phenyl)urea (Gedatolisib), (Z)-5-((4-(pyridin-4-yl)quinolin-6-yl)methylene)thiazolidine-2,4-dione (GSK1059615), (S)-1-(4-((2-(2-aminopyrimidin-5-yl)-7-methyl-4-morpholinothieno[3,2-d]pyrimidin-6-yl)methyl)piperazin-1-yl)-2-hydroxypropan-1-one (Apitolisib), 2-amino-8-((1r,4r)-4-(2-hydroxyethoxy)cyclohexyl)-6-(6-methoxypyridin-3-yl)-4-methylpyrido[2,3-d]pyrimidin-7(8H)-one (PF-04691502), (R)-2-(1-(7-methyl-2-morpholino-4-oxo-4H-pyrido[1,2-a]pyrimidin-9-yl)ethylamino)benzoic acid (AZD6482), N-(2,3-dihydro-7,8-dimethoxyimidazo[1,2-c]quinazolin-5-yl)-3-pyridinecarboxamide (PIK-90), 2,4-difluoro-N-(2-methoxy-5-(4-(pyridazin-4-yl)quinolin-6-yl)pyridin-3-yl)benzenesulfonamide (Omipalisib), N-[5-[4-Chloro-3-[(2-hydroxyethyl)sulfamoyl]phenyl]-4-methylthiazol-2-yl]acetamide (PIK-93), N-[4-[[[3-[(3,5-dimethoxyphenyl)amino]-2-quinoxalinyl]amino]sulfonyl]phenyl]-3-methoxy-4-methyl-benzamide (Voxtalisib), (2S)-N1-(5-(2-tert-butylthiazol-4-yl)-4-methylthiazol-2-yl)pyrrolidine-1,2-dicarboxamide (A66), (E)-N′-((6-bromoH-imidazo[1,2-a]pyridin-3-yl)methylene)-N,2-dimethyl-5-nitrobenzenesulfonohydrazide hydrochloride (PIK-75 HCl), (Z)-5-(quinoxalin-6-ylmethylene)thiazolidine-2,4-dione (AS-605240), N-1-[4-methyl-5-[2-(2,2,2-trifluoro-1,1-dimethylethyl)-4-pyridinyl]-2-thiazolyl]-(2S)-1,2-pyrrolidinedicarboxamide, (Alpelisib), 2-(difluoromethyl)-1-(4,6-dimorpholino-1,3,5-triazin-2-yl)-1H-benzo[d]imidazole (ZSTK474), 1-[[4′-(cyclopropylmethyl)-2-(4-pyridinylamino)[4,5′-bipyrimidin]-2′-yl]amino]-2-methyl-2-propanol (VPS34 inhibitor 1), 2-[(1S)-1-[4-amino-3-[3-fluoro-4-(1-methylethoxy)phenyl]-1H-pyrazolo[3,4-d]pyrimidin-1-yl]ethyl]-6-fluoro-3-(3-fluorophenyl)-4H-1-Benzopyran-4-one (TGR-1202), (8S)-9-[(5-chloro-3-pyridinyl)methyl]-6,7,8,9-tetrahydro-2-[(3R)-3-methyl-4-morpholinyl]-8-(trifluoromethyl)-4H-pyrimido[1,2-a]pyrimidin-4-one (SAR405), (2S)-2-[[5,6-dihydro-2-[1-(1-methylethyl)-1H-1,2,4-triazol-5-yl]imidazo[1,2-d][1,4]benzoxazepin-9-yl]oxy]-propanamide (GDC-0326), 3-(2,3-dihydro-1,4-benzodioxin-6-yl)-5-(4-morpholinyl)-7H-thieno[3,2-b]pyran-7-one (SF2523), 2-amino-N-[(1S)-1-[1,2-dihydro-8-[2-(1-methyl-1Hpyrazol-4-yl)ethynyl]-1-oxo-2-phenyl-3-isoquinolinyl]ethyl]-pyrazolo[1,5-a]pyrimidine-3-carboxamide (IPI-549), 5-(2,6-dimorpholinopyrimidin-4-yl)-4-(trifluoromethyl)pyridin-2-amine (Buparlisib), etc. The biochemistry is relatively mature providing substrates and tools that a skilled artisan would have available to modify for a best balance between safety and effectiveness.

SP600125 is a broad-spectrum JNK inhibitor for JNK1, JNK2 and JNK3. 1,4-Diamino-2,3-dicyano-1,4-bis(2-aminophenylthio)-butadiene (U0106) an MEK1 and MEK2 Inhibitor.

Bafilomycin A1 ((3Z,5E,7R,8S,9S,11E,13E,15S,16R)-8-Hydroxy-16-[(1S,2R,3S)-2-hydroxy-1-methyl-3-[(2R,4R,5S,6R)-tetrahydro-2,4-dihydroxy-5-methyl-6-(1-methylethyl)-2H-pyran-2-yl]butyl]-3,15-dimethoxy-5,7,9,11-tetramethyloxacyclohexadeca-3,5,11,13-tetraen-2-one (C₃₅H₅₈O₉). Bafilomycin A1 belongs to a group of macrolide 16-membered lactone ring antibiotics produced by Streptomyces griseus Sulphurus ssp. It is an inhibitor of the late phase of autophagy by its inhibition of vacuolar H⁺ ATPase (V-ATPase) to prevent maturation of autophagic vacuoles (by inhibiting fusion between autophagosomes and lysosomes).

Wortmannin is a cell-permeable, fungal metabolite that acts as a potent, selective and essentially irreversible inhibitor of phosphatidylinositol 3-kinase (PI3K). There is increasing evidence of the involvement of PI3K in Toll-like receptor (TLR) signaling. Inhibition of PI3K with wortmannin enhances TLR-mediated inducible nitric-oxide synthase (iNOS) expression, activates NF-κB and up-regulates cytokine mRNA production. Furthermore, PI3K is required for autophagy. Autophagy is a complex pathway in which cell material can be sequestered and delivered to the lysosome for degradation. Inhibition of PI3K with wortmannin can inhibit autophagic sequestration.

SB203580 is a pyridinyl imidazole inhibitor widely used to elucidate the roles of p38 mitogen-activated protein (MAP) kinase. SB203580 inhibits also the phosphorylation and activation of protein kinase B (PKB, also known as Akt). Both kinases are involved in a wide array of signaling pathways, including the TLR signaling pathway. Moreover, several studies suggest that p38 MAPKs regulate distinct phases of autophagy. p38 can elicit autophagy via Beclin1. Contrarily, p38a has also been reported to inhibit autophagy by interfering with the trafficking of Atg9.

4-(4-Fluorophenyl)-2-(4-hydroxyphenyl)-5-(4-pyridyl)-1H-imidazole (SB202190) MAP Kinase Inhibitor—p38/RK MAP Kinase Inhibitor—Autophagy inducer SB202190, a close relative of SB203580, is widely used to assess the physiological roles of p38a and p3813 MAPKs. Recent studies have identified other protein kinases including: GAK, CK1 and RIP2 that are potently inhibited by SB202190 (as well as SB203580). Further, SB202190 was shown to induce autophagic vacuoles through cross-inhibition of the PI3K/mTOR pathway.

2-(4-morpholino)-8-phenyl-4H-1-benzopyran-4-one (LY294002) is a potent, cell permeable inhibitor of phosphatidylinositol 3-kinase (PI3K) that acts on the ATP binding site of the enzyme. The PI3K pathway is extensively studied for its role in inhibiting apoptosis. PI3K is also known to regulate TLR-mediated inflammatory responses. Furthermore, PI3K is required for autophagy. Autophagy is a complex pathway in which cell material can be sequestered and delivered to the lysosome for degradation. Inhibition of PI3K with LY294002 can inhibit autophagic sequestration.

3-methyladenine (3-MA) is an inhibitor of phosphatidylinositol 3-kinases (PI3K). PI3K plays an important role in many biological processes, including controlling the activation of mTOR, a key regulator of autophagy. 3-MA inhibits autophagy by blocking autophagosome formation via the inhibition of class III PI3K [1]. 3-MA plays a dual role in autophagy. Prolonged treatment with 3-MA promotes autophagy under nutrient-rich conditions, whereas 3-MA inhibits starvation-induced autophagy. These results are attributed to differential effects on class I versus class III PI3K. In addition to its role in autophagy, 3-MA has been implicated in cancer therapy. It has been revealed that 3-MA suppresses the invasion of highly metastatic cancer cells through the inhibition of class I and II PI3K. Further studies demonstrated that 3-MA can induce caspase-dependent cell death that is independent of autophagy inhibition.

2-methyl-2-{4-[3-methyl-2-oxo-8-(quinolin-3-yl)-2,3-dihydro-1H-imidazo[4,5-c]quinolin-1-yl]phenyl}propanenitrile (Dactolisib) (codenamed NVP-BEZ235 and BEZ-235) is an imidazoquinoline derivative acting as a PI3K inhibitor. It also inhibits mTOR.

2-(1H-indazol-4-yl)-6-((4-(methylsulfonyl)piperazin-1-yl)methyl)-4-morpholinothieno[3,2-d]pyrimidine (Pictilisib) (GDC-0941) is a potent inhibitor of PI3Kα/δ.

(S)-2-(1-(9H-purin-6-ylamino)propyl)-5-fluoro-3-phenylquinazolin-4(3H)-one (Idelalisib) (CAL-101, GS-1101) is a selective p110δ inhibitor.

(S)-2-(1-(9H-purin-6-ylamino)propyl)-5-fluoro-3-phenylquinazolin-4(3H)-one (AZD8186) is a potent and selective inhibitor of PI3Kβ and PI3Kδ.

(R)-8-(1-(3,5-difluorophenylamino)ethyl)-N,N-dimethyl-2-morpholino-4-oxo-4H-chromene-6-carboxamide (AZD8186) is a potent and selective inhibitor of PI3Kβ and PI3K.

L-serine, L-arginyl-L-glutaminyl-L-isoleucyl-L-lysyl-L-isoleucyl-L-tryptophyl-L-phenylalanyl-L-glutaminyl-L-asparaginyl-L-arginyl-L-arginyl-L-methionyl-L-lysyl-L-tryptophyl-L-lysyl-L-lysyl-L-seryl-L-aspartylglycylglycyl-O- phosphono-L-tyrosyl-L-methionyl-L-aspartyl-L-methionyl-(740Y-P) is as effective as a growth factor (FGF2) at promoting neuronal cell survival via the established PI 3-kinase-Akt survival cascade.

4-(4-cyano-2-fluorophenyl)-2-morpholino-5-(2H-1,2,4-triazol-3-yl)thiophene-3-carbonitrile (PF-4989216) is a potent and selective PI3K inhibitor.

N-(3-{[(3-{[2-chloro-5-(methoxy)phenyl]amino}quinoxalin-2-yl)amino]sulfonyl}phenyl)-2-methylalaninamide (Pilaralisib) (XL147) is a selective and reversible class I PI3K inhibitor for PI3Kα/δ/γ.

6-((4-(cyclopropylmethyl)piperazin-1-yl)methyl)-2-(5-fluoro-1H-indol-4-yl)-4-morpholinothieno[3,2-d]pyrimidine (PI-3065) is a selective p110δ inhibitor.

4′-(cyclopropylmethyl)-N2-4-pyridinyl-[4,5′-Bipyrimidine]-2,2′-diamine (PIK-III), which is a selective inhibitor of VPS34 enzymatic activity, inhibits autophagy and de novo lipidation of LC3 and leads to the stabilization of autophagy substrates.

1-[[2-[(2-chloro-4-pyridinyl)amino]-4′-(cyclopropylmethyl)[4,5′-bipyrimidin]-2′-yl]amino]-2-methyl-2-Propanol (VPS34-1N1) is a potent and highly selective Vps34 inhibitor with IC50 of 25 nM in vitro, which does not significantly inhibit the isoforms of class I as well as class II PI3Ks.

5-(6-(3-methoxyoxetan-3-yl)-7-methyl-4-morpholinothieno[3,2-d]pyrimidin-2-yl)pyrimidin-2-amine (GNE-317) is a potent, brain-penetrant PI3K inhibitor.

2-amino-8-ethyl-4-methyl-6-(1H-pyrazol-5-yl)pyrido[2,3-d]pyrimidin-7-one (Voxtalisib) (SAR245409, XL765) is a dual inhibitor of mTOR/PI3K, mostly for p110γ with IC50 of 9 nM; also inhibits DNA-PK and mTOR.

2-(3,4-dihydroxyphenyl)-3,5,7-trihydroxy-4H-1-Benzopyran-4-one (Quercetin), a natural flavonoid present in vegetables, fruit and wine, is a stimulator of recombinant SIRT1 and also a PI3K inhibitor.

Ethyl 6-(5-(phenylsulfonamido)pyridin-3-yl)imidazo[1,2-a]pyridine-3-carboxylate (HS-173) is a potent PI3Kα inhibitor.

N-[5-[4-[5-[[(2R,6S)-2,6-dimethyl-4-morpholinyl]methyl]-2-oxazolyl]-1H-indazol-6-yl]-2-methoxy-3-pyridinyl]-methanesulfonamide (GSK2292767) is a potent and selective PI3Kδ inhibitor.

N-((S)-1-(7-fluoro-2-(pyridin-2-yl)quinolin-3-yl)ethyl)-9H-purin-6-amine (AMG319) is a potent and selective PI3Kδ inhibitor with IC50 of 18 nM, >47-fold selectivity over other PI3Ks.

5-(2-amino-8-fluoro[1,2,4]triazolo[1,5-a]pyridin-6-yl)-N-(1,1-dimethylethyl)-3-pyridinesulfonamide (CZC24832) is the first selective PI3Kγ inhibitor with IC50 of 27 nM, with 10-fold selectivity over PI3Kβ and >100-fold selectivity over PI3Kα and PI3Kδ.

α,α-dimethyl-4-[5,6-dihydro-2-[3-methyl-1-(1-methylethyl)-1H-1,2,4-triazol-5-yl]imidazo[1,2-d][1,4]benzoxazepin-9-yl]-1H-pyrazole-1-acetamide (Taselisib) (GDC 0032) is a potent, next-generation β isoform-sparing PI3K inhibitor targeting PI3Kα/δ/γ.

5-[8-methyl-9-(1-methylethyl)-2-(4-morpholinyl)-9H-purin-6-yl]-2-pyrimidinamine (VS-5584) (SB2343) is a potent and selective dual PI3K/mTOR inhibitor for mTOR, PI3Kα/β/δ/γ.

3-(2,4-diamino-6-pteridinyl)-phenol (TG100713) is a pan-PI3K inhibitor against PI3Kγ, PI3Kδ, PI3Kα and PI3Kβ.

1-(4-(3-ethyl-7-morpholino-3H-[1,2,3]triazolo[4,5-d]pyrimidin-5-yl)phenyl)-3-(4-(1-methylpiperazine-4-carbonyl)phenyl)urea (PKI-402) is a potent dual pan-PI3K/mTOR inhibitor targeting PI3Kα/β/γ/δ and mTOR; also potent to PI3Kα mutants E545K and H1047R.

2-((4-amino-1H-pyrazolo[3,4-d]pyrimidin-1-yl)methyl)-5-methyl-3-o-tolylquinazolin-4(3H)-one (PIK-293) is a PI3K inhibitor, mostly for PI3Kδ.

(E)-5-((5-(4-fluorophenyl)furan-2-yl)methylene)thiazolidine-2,4-dione (CAY10505) is dehydroxyl of AS-252424, which is a PI3Kγ inhibitor.

5-(7-(methylsulfonyl)-2-morpholino-6,7-dihydro-5H-pyrrolo[2,3-d]pyrimidin-4-yl)pyrimidin-2-amine (CH5132799) inhibits class I PI3Ks, particularly PI3Kα.

6-amino-N-[3-[4-(4-morpholinyl)pyrido[3′,2′:4,5]furo[3,2-d]pyrimidin-2-yl]phenyl]-3-Pyridinecarboxamide (YM201636) is a selective PIKfyve inhibitor.

5-pyrimidinecarboxamide, 2-amino-N-[2,3-dihydro-7-methoxy-8-[3-(4-morpholinyl)propoxy]imidazo[1,2-c]quinazolin-5-yl]- (Copanlisib) (BAY 80-6946) is a potent pan-class I PI3K.

(Z)-5-((2,2-difluorobenzo[d][1,3]dioxol-5-yl)methylene)thiazolidine-2,4-dione (AS-604850) is a selective, ATP-competitive PI3Kγ inhibitor.

2-((4-amino-3-(3-hydroxyphenyl)-1H-pyrazolo[3,4-d]pyrimidin-1-yl)methyl)-5-methyl-3-otolylquinazolin-4(3H)-one (PIK-294) is a highly selective p110δ inhibitor.

N-hydroxy-2-(((2-(6-methoxypyridin-3-yl)-4-morpholinothieno[3,2-d]pyrimidin-6-yl)methyl)(methyl)amino)pyrimidine-5-carboxamide (CUDC-907) is a dual PI3K and HDAC inhibitor for PI3Kα and HDAC1/2/3/10.

2-methyl-1-[[2-methyl-3-(trifluoromethyl)phenyl]methyl]-6-(4-morpholinyl)-1H-Benzimidazole-4-carboxylic acid (GSK2636771) is a potent, orally bioavailable and selective inhibitor of PI3Kβ.

8-(6-methoxypyridin-3-yl)-3-methyl-1-(4-(piperazin-1-yl)-3-(trifluoromethyl)phenyl)-1Himidazo[4,5-c]quinolin-2(3H)-one maleic acid (BGT226) (NVP-BGT226) is a novel class I PI3K/mTOR inhibitor for PI3Kα/β/γ.

(Z)-5-((5-(4-fluoro-2-hydroxyphenyl)furan-2-yl)methylene)thiazolidine-2,4-dione (AS-252424) is a novel, potent PI3Kγ inhibitor.

6,7-bis(3-hydroxyphenyl)pteridine-2,4-diamine (TG100-115) is a PI3Kγ/δ inhibitor.

8-chloro-2-phenyl-3-[(1S)-1-(9H-purin-6-ylamino)ethyl]-1(2H)-Isoquinolinone (Duvelisib) (IPI-145, INK1197) is a novel and selective PI3Kδ/γ inhibitor.

1-(4-(4-(dimethylamino)piperidine-1-carbonyl)phenyl)-3-(4-(4,6-dimorpholino-1,3,5-triazin-2-yl)phenyl)urea (Gedatolisib) (PF-05212384, PKI-587) is a highly potent dual inhibitor of PI3Kα, PI3Kγ and mTOR.

(Z)-5-((4-(pyridin-4-yl)quinolin-6-yl)methylene)thiazolidine-2,4-dione (GSK1059615) is a dual inhibitor of PI3Kα/β/δ/γ(reversible) and mTOR.

(S)-1-(4-((2-(2-aminopyrimidin-5-yl)-7-methyl-4-morpholinothieno[3,2-d]pyrimidin-6-yl)methyl)piperazin-1-yl)-2-hydroxypropan-1-one (Apitolisib) (GDC-0980, RG7422) is a potent, class I PI3K inhibitor for PI3Kα/β/δ/γ.

2-amino-8-((1r,4r)-4-(2-hydroxyethoxy)cyclohexyl)-6-(6-methoxypyridin-3-yl)-4-methylpyrido[2,3-d]pyrimidin-7(8H)-one (PF-04691502) is an ATP-competitive PI3K(α/β/δ/γ)/mTOR dual inhibitor.

(R)-2-(1-(7-methyl-2-morpholino-4-oxo-4H-pyrido[1,2-a]pyrimidin-9-yl)ethylamino)benzoic acid (AZD6482) is a PI3Kβ inhibitor.

N-(2,3-dihydro-7,8-dimethoxyimidazo[1,2-c]quinazolin-5-yl)-3-pyridinecarboxamide (PIK-90) is a PI3Kα/γ/δ inhibitor.

2,4-difluoro-N-(2-methoxy-5-(4-(pyridazin-4-yl)quinolin-6-yl)pyridin-3-yl)benzenesulfonamide (Omipalisib) (GSK2126458, GSK458) is a highly selective and potent inhibitor of p110α/β/δ/γ, mTORC1/2.

N-[5-[4-Chloro-3-[(2-hydroxyethyl)sulfamoyl]phenyl]-4-methylthiazol-2-yl]acetamide (PIK-93) is the first potent, synthetic P14K (PI4KIIIβ) inhibitor.

N-[4-[[[3-[(3,5-dimethoxyphenyl)amino]-2-quinoxalinyl]amino]sulfonyl]phenyl]-3-methoxy-4-methyl-benzamide (Voxtalisib (SAR245409, XL765) Analogue) is a dual inhibitor of mTOR/PI3K, mostly for p110γ.

(2S)-N1-(5-(2-tert-butylthiazol-4-yl)-4-methylthiazol-2-yl)pyrrolidine-1,2-dicarboxamide (A66) is a potent and specific p110a inhibitor.

(E)-N′-((6-bromoH-imidazo[1,2-a]pyridin-3-yl)methylene)-N,2-dimethyl-5-nitrobenzenesulfonohydrazide hydrochloride (PIK-75 HCl) is a p110α inhibitor. It also potently inhibits DNA-PK.

(Z)-5-(quinoxalin-6-ylmethylene)thiazolidine-2,4-dione (AS-605240) selectively inhibits PI3Kγ.

N-1-[4-methyl-5-[2-(2,2,2-trifluoro-1,1-dimethylethyl)-4-pyridinyl]-2-thiazolyl]-(2S)-1,2-Pyrrolidinedicarboxamide, (Alpelisib) (BYL719) is a potent and selective PI3Kα inhibitor.

2-(difluoromethyl)-1-(4,6-dimorpholino-1,3,5-triazin-2-yl)-1H-benzo[d]imidazole (ZSTK474) inhibits class I PI3K isoforms.

N-(3-(benzo[c][1,2,5]thiadiazol-5-ylamino)quinoxalin-2-yl)-4-methylbenzenesulfonamide (XL147 analogue) (SAR245408) is a selective and reversible class I PI3K inhibitor for PI3Kα/δ/γ. See Pilaralisib.

2-((6-amino-9H-purin-9-yl)methyl)-5-methyl-3-o-tolylquinazolin-4(3H)-one (IC-87114) is a selective PI3Kδ inhibitor.

7-methyl-2-morpholino-9-(1-(phenylamino)ethyl)-4H-pyrido[1,2-a]pyrimidin-4-one (TGX-221) is a p110β-specific inhibitor.

8-(4-dibenzothienyl)-2-(4-morpholinyl)-4H-1-benzopyran-4-one (NU7441) (KU-57788) is a highly potent and selective DNA-PK inhibitor.

3-[4-(4-morpholinyl)pyrido[3′,2′:4,5]furo[3,2-d]pyrimidin-2-yl]-phenol (PI-103) is a multi-targeted PI3K inhibitor for p110α/β/δ/γ.

1,3-dihydro-8-[5-(1-hydroxy-1-methylethyl)-3-pyridinyl]-1-[(2S)-2-methoxypropyl]-3-methyl-2H-imidazo[4,5-c]quinolin-2-one (LY3023414) is an oral ATP competitive inhibitor of the class I PI3K isoforms, mTOR and DNA-PK.

1-[4-[5-[5-amino-6-[5-(1,1-dimethylethyl)-1,3,4-oxadiazol-2-yl]-2-pyrazinyl]-1-ethyl-

1H-1,2,4-triazol-3-yl]-1-piperidinyl]-3-hydroxy-1-propanone (AZD8835) is a novel mixed inhibitor of PI3Kα and PI3Kδ

5-[8,9-dihydro-6,6-dimethyl-4-(4-morpholinyl)-6H-[1,4]oxazino[4,3-e]purin-2-yl]-2-pyrimidinamine, (GDC-0084) is a brain penetrant inhibitor of PI3K and mTOR.

6-(1H-indol-4-yl)-4-[5-[[4-(1-methylethyl)-1-piperazinyl]methyl]-2-oxazolyl]-1H-Indazole (Nemiralisib) (GSK-2269557) is a potent and selective PI3Kδ inhibitor.

1-[[4′-(cyclopropylmethyl)-2-(4-pyridinylamino)[4,5′-bipyrimidin]-2′-yl]amino]-2-methyl-2-propanol (VPS34 inhibitor 1) (Compound 19, PIK-III analogue) is a potent and selective inhibitor of VPS34.

2-[(1S)-1-[4-amino-3-[3-fluoro-4-(1-methylethoxy)phenyl]-1H-pyrazolo[3,4-d]pyrimidin-1-yl]ethyl]-6-fluoro-3-(3-fluorophenyl)-4H-1-Benzopyran-4-one (TGR-1202) is considered a novel or next generation PI3Kδ inhibitor. It inhibits PI3Kδ activity.

(8S)-9-[(5-chloro-3-pyridinyl)methyl]-6,7,8,9-tetra hydro-2-[(3R)-3-methyl-4-morpholinyl]-8-(trifluoromethyl)-4H-pyrimido[1,2-a]pyrimidin-4-one (SAR405) is a highly selective, low-molecular-mass kinase inhibitor of Vps34 (KD 1.5 nM) and no effect up to at least about 10 μM on class I and class II PI3Ks and on mTOR.

(2S)-2-[[5,6-dihydro-2-[1-(1-methylethyl)-1H-1,2,4-triazol-5-yl]imidazo[1,2-d][1,4]benzoxazepin-9-yl]oxy]-propanamide (GDC-0326) is also a potent, selective inhibitor but on PI3Kα, with Ki value of 0.2 nM with high selectivity over the other class I isoforms.

3-(2,3-dihydro-1,4-benzodioxin-6-yl)-5-(4-morpholinyl)-7H-thieno[3,2-b]pyran-7-one (SF2523) is a highly selective and potent inhibitor of PI3K with IC50 values of 34 nM, 158 nM, 9 nM, 241 nM and 280 nM for PI3Kα, PI3Kγ, DNA-PK, BRD4 and mTOR, respectively.

2-amino-N-[(1S)-1-[1,2-dihydro-8-[2-(1-methyl-1H pyrazol-4-yl)ethynyl]-1-oxo-2-phenyl-3-isoquinolinyl]ethyl]-pyrazolo[1,5-a]pyrimidine-3-carboxamide (IPI-549) is a potent inhibitor of PI3K-γ with >100-fold selectivity over other lipid and protein kinases.

5-(2,6-dimorpholinopyrimidin-4-yl)-4-(trifluoromethyl)pyridin-2-amine (Buparlisib) (BKM120, NVP-BKM120) is a selective PI3K inhibitor of p110a/3/6/γ.

One embodiment of the invention relates to modulating undesired immune reactions in a person by addressing the autophagic/mitophagic processes to arrest or reverse the disease processes. The person who would benefit from the featured intervention might be a person diagnosed as having or having had an autoimmune event, a person experiencing or having experienced a harmful or an unwanted immune event (e.g., an inflammatory event), a person known to have or be suspected of having (inherited from a parent) a CLEC16A locus variant, and/or a person who evidence or opinion instructs would benefit from modulated mitophagy/autophagy metabolism.

Practicing one embodiment of the invention would feature delivering, administering providing or otherwise delivering a therapeutic substance in accordance with this invention to such person.

As an adjunct to the therapies described herein conventional treatments for pain such as NSAIDS for both their anti-pain and anti-inflammatory effects, may be delivered concurrent with, as a prelude to, as a follow-up or as alternating therapy. Activation of the TH-2 anti-inflammatory pathway may serve similarly in an adjunct status. The multi-pronged attack with controlled apoptosis and a back-up therapy to suppress inflammation optimizes effects of each therapeutic strategy meaning each therapy in concert is more effective than each individually.

Activities of anti-inflammatory cytokines including, but not limited to: IL-4, IL-9, IL-10, IL-11, IL-12, IL-13, IL-19, TGFβ, etc., may also be applied in some embodiments as adjunct therapy.

These and other interventions with general anti-inflammatory results, such as: inducing and/or activating STAT1, STAT3, STAT5, STAT6, SOCS-3, IκB, BCL3 and/or inhibiting activity and/or expression of p38, NF-κB, p44/42, HuR, Egr-1, and the like, can serve with adjunct therapeutic benefit.

One example preliminarily involves activation of the cannabinoid receptor, CB₂, which results in upregulation of STAT5 and induces synthesis and release of the anti-inflammatory interleukin, IL-4 and also IL-13 in some cells. These cytokines then induce CB₁ cannabinoid receptor that when activated suppresses pro-inflammatory activities of cytokines such as IL-2.

Examples of active cannabinoids include, but are not limited to: cannabigerolic acid (CBGA) (antibiotic); cannabigerolic acid monomethylether (CBGAM); cannabigerol (CBG) (antibiotic, antifungal, anti-inflammatory, analgesic); Cannabigerol monomethylether (CBGM); cannabigerovarinic acid (CBGVA); Cannabigerovarin (CBGV); Cannabichromenic acid (CBCA); Cannabichromene (CBC) (antibiotic, antifungal, anti-inflammatory, analgesic); Cannabichromevarinic acid (CBCVA); Cannabichromevarin (CBCV); Cannabidiolic acid (CBDA) (antibiotic); Cannabidiol (CBD) ((antioxidant, anxiolytic, antispasmodic, anti-inflammatory, analgesic); cannabidiol monomethylether (CBDM); cannabidiol C₄ (CBD-C4); cannabidivarinic acid (CBDVA); cannabidivarin (CBDV); cannabidiorcol (CBD-C1); Δ⁹-tetrahydrocannabinolic acid A (THCA-A); Δ⁹-tetrahydrocannabinolic acid B (THCA-B); 6a,10a-trans-6a,7,8,10a-tetrahydro-6,6,9-trimethyl-3-pentyl-6H-dibenzo[b,d]pyran-1-ol, (Δ⁹-tetrahydrocannabinol, THC) (analgesic, antioxidant, antiemetic, anti-inflammation); Δ⁹-tetrahydrocannabinolic acid-C4 (THCA-C4); Δ⁹-tetrahydrocannabinol-C4 (THC-C4); Δ⁹-tetrahydrocannabivarinic acid (THCVA); Δ⁹-tetrahydrocannabivarinic (THCV); Δ⁷-cis-isotetrahydrocannabivarin; Δ⁹-tetrahydrocannabiorcolic acid (THCA-C1); tetrahydrocannabiorcol (THC-C1); Δ⁸-tetrahydrocannabinolic acid (Δ⁸-TCA); Δ⁸-tetrahydrocannabinol (Δ⁸-THC); cannabicyclol (CBL); cannabicyclolicacid (CBLA); cannabicyclovarin (CBLV), cannabiesoic acid A (CBEA-A); cannabiesoic acid B (CBEA-B); cannabieson (CBE); cannabinolic acid (CBNA); cannabinol (CBN); cannabinol methylether (CBNM); cannabinol-C4 (CBN-C4); cannabivarin (CBV); cannabinol-C2 (CBN-C2); cannabiorcol (CBN-C1); cannabinodiol (CBND); cannabinidivarin (CBDV); cannabitriol (CBT); 10-Ethoxy-9-hydroxy-Δ^{6a}-tetrahydrocannabinol (10-EHDT); 8,9-dihydroxy-delta-6a-tetrahydrocannabinol (8,9-DHDT); cannabitriolvarin (CBTV); ethoxy-cannabitriolvarin (CBTVE); dehydrocannabifuran (DCBF); cannabifuran (CBF); cannabichromanon (CBCN); cannabicitran (CBT); 10-oxo-Δ-6a-tetrahydrocannabinol (OTHC); Δ⁹-cis-tetrahydrocannabinol (cis-THC); 3,4,5,6-tetrahydro-7-hydroxy-α-α-2-trimethyl-9-n-propyl-2,6-methano-2H-1-benzoxocin-5-methanol (2H-iso-HHCV); cannabiripsol (CBR); trihydroxy-Δ⁹-tetrahydrocannabinol (triOH-THC), etc.

Maintaining anti-inflammatory activities may also be accomplished by inhibiting the major enzyme for metabolizing anti-inflammatory cannabinoids, FAAH. Oleoylethanolamide (OEA), palmitoylethanolamide (PEA), and linoleoylethanolamide (LEA) are exemplary FAAH inhibitors. Also N-alkylamides exert selective effects on the CB₂, which have been shown to exert anti-inflammatory effects similar to anandamide (AEA). Echinacea contains multiple N-alkylamides that may act through this mechanism.

The NSAIDS and similar small molecules may include one or more of the following non-exclusive list: sulindac, sulindac sulfide, pravadoline, naproxen, naproxen sodium salt, meclofenamate sodium, ibupropfen, S-ibuprofen, piroxicam, ketoprofen, S-ketoprofen, R-ibuprofen, Ebselen, ETYA, diclofenac, diclofenac diethylamine, flurbiprofen, fexofenadine, Pterostilbene, Pterocarpus marsupium, 9,12-octadecadiynoic acid, Ketorolac (tromethamine salt), NO-indomethacin, S-flurbiprofen, sedanolide, green tea extract (e.g., epicatechin), licofelone, lornoxicam, rac ibuprofen-d3, ampirxicam, zaltoprofen, 7-(trifluoromethyl)1H-indole-2,3-dione, aceclofenac, acetylsalicylic acid-d4, S-ibuprofen lysinate, loxoprofen, CAY10589, ZU-6, isoicam, dipyrone, YS121, and MEG (mercaptoethylguanidine).

Natural biomolecules such as interleukin (IL)-1 receptor antagonist, IL-4, IL-6, IL-10, IL-11, IL-13, precursors, active fragments and/or derivatives, preferably derived from the recipient species may also be advantageously supplemented to assist the present invention in its inflammation control, e.g., specific cytokine receptors for IL-1, tumor necrosis factor-α, and IL-18 may also function as pro-inflammatory cytokine inhibitors.

The form of the therapeutic substance(s) is not limited. Depending on the specific chemistry, it may be a liquid; it may be dissolved in a liquid; it may be in the form of a paste or gel; it may be in the form of a suppository; it may be formulated as a transdermal patch; it may be formulated as a nasal spray; it may be formulated for buccal administration; it may be delivered IM, SC or IV; It may be made as a gelcap; it may be pressed into a pill; it may be delivered as a powder; It may be enclosed in a capsule; it may be formulated in any manner suitable for delivering such intervention treatment to a person. It may even be formulated for veterinary purposes.

The substance to be delivered is preferably a compound or composition designed for a balance of safety and efficacy relating to the autophagic/mitophagic activity which modulation would benefit a person or persons for whom the substance is intended. Select embodiments may include one or a plurality of active ingredient substances in combination with pharmaceutical excipients appropriate for the intended mode of delivery, application or administration. Preferred substance(s) may comprise chloroquine and or a chloroquine derivative modified for improved efficacy, safety, ability to deliver to the intended person wherever they might be, shelf-life and/or any other desired characteristic.

Several scores of candidate active ingredient components are described above. Several of these will not be appropriate for every conceived mode of delivery. But the breadth of example compounds is instructive to the skilled artisan of the types, classes, characteristics and reactivities easily formulated to meet the purposes and/or results expressed in this patent.

While chloroquine may possibly be the active substance with the greatest past experience, other compounds formulated to meet the outcomes targeted in the present invention may be more appropriate for any one intended use. Thus the scope of the invention is clearly of a breadth to include all compounds listed and discussed above as well as esters, salts, amines, oxides, hydroxyls, hydroxides, amides, chelates, biosimilars, extensions, truncations, when relevant and the like derivations used in the food and pharmaceutical arts.

Another embodiment of the invention is the actual preparation or formulation of the substance to be delivered. In a preferred embodiment, a medicament is formulated for the purpose of ameliorating an overzealous immune activity in a person or animal. The overzealous activity may involve autoimmune activity, inflammation, etc. The substance would be one formulated in any suitable format for delivery to modify at least one of the autophagy/mitophagy pathways. A preferred embodiment modifies a pathway involving PI3K, more preferably modifying activity of PI3K, and may be intended to modify such pathway directly or indirectly, e.g., by modulating a serial or parallel pathway, by modifying availability of any ligand or substrate in the path, by modifying availability or transportability or delivery mechanisms of any involved macro or biomolecule, small molecule or ion.

1. A method of arresting disease causing auto immune activity, said method comprising delivering to at least one cell of a person or animal who stands to benefit, at least one substance that modulates autophagic and/or mitophagic activity within said at least one cell.

2. The method of claim 1 wherein said person or animal who stands to benefit has a recognized or diagnosed inflammatory or autoimmune disorder.

3. The method of claim 2 wherein said inflammatory or autoimmune disorder is selected from the group consisting of: type 1 diabetes, multiple sclerosis, inflammatory bowel disease, psoriasis, Lupus, primary biliary cirrhosis and asthma.

4. The method of claim 1 wherein said delivery is to a person and said person who stands to benefit carries a variant sequence of DNA corresponding to the CLEC16A locus.

5. The method of claim 4 wherein said variant is selected from the group consisting of: rs12708716 and rs12927355.

6. The method of claim 1 wherein said at least one substance is selected from the group consisting of: compounds that inhibit activity of the PI3K pathway.

7. The method of claim 1 wherein said at least one substance is selected from the group consisting of: N4-(7-chloro-4-quinolinyl)-N1,N1-dimethyl-1,4-pentanediamine (chloroquine), 2H-dibenzo[cd,g]indazol-6-one (SP600125), 1,4-diamino-2,3-dicyano-1,4-bis(2-aminophenylthio)-butadiene (U0106), Bafilomycin A1 ((3Z,5E,7R,8S,9S,11E,13E,15S,16R)-8-hydroxy-16-[(1S,2R,3S)-2-hydroxy-1-methyl-3-[(2R,4R,5S,6R)-tetra hydro-2,4-dihydroxy-5-methyl-6-(1-methylethyl)-2H-pyran-2-yl]butyl]-3,15-dimethoxy-5,7,9,11-tetramethyloxacyclohexadeca-3,5,11,13-tetraen-2-one (C₃₅H₅₈O₉), (1S,6bR,9aS,11R,11bR)-11-(acetyloxy)-1,6b,7,8,9a,10,11,11b-octahydro-1-(methoxymethyl)-9a,11b-dimethyl-3H-furo[4,3,2-de]indeno[4,5-h]-2-benzopyran-3,6,9-trione (Wortmann in), 4-(4-(4-fluorophenyl)-2-(4-(methylsulfinyl)phenyl)-1H-imidazol-5-yl)pyridine (SB203580), 4-(4-fluorophenyl)-2-(4-hydroxyphenyl)-5-(4-pyridyl)-1H-imidazole (SB202190), 2-(4-morpholino)-8-phenyl-4H-1-benzopyran-4-one (LY294002), 3-methyladenine (3-MA), 2-Methyl-2-{4-[3-methyl-2-oxo-8-(quinolin-3-yl)-2,3-dihydro-1H-imidazo[4,5-c]quinolin-1-yl]phenyl}propanenitrile (Dactolisib), 2-(1H-indazol-4-yl)-6-((4-(methylsulfonyl)piperazin-1-yl)methyl)-4-morpholinothieno[3,2-d]pyrimidine (Pictilisib), (S)-2-(1-(9H-purin-6-ylamino)propyl)-5-fluoro-3-phenylquinazolin-4(3H)-one (Idelalisib), (S)-2-(1-(9H-purin-6-ylamino)propyl)-5-fluoro-3-phenylquinazolin-4(3H)-one (AZD8186), (R)-8-(1-(3,5-difluorophenylamino)ethyl)-N, N-dimethyl-2-morpholino-4-oxo-4H-chromene-6-carboxamide (AZD8186), L-serine, L-arginyl-L-glutaminyl-L-isoleucyl-L-lysyl-L-isoleucyl-L-tryptophyl-L-phenylalanyl-L-glutaminyl-L-asparaginyl-L-arginyl-L-arginyl-L-methionyl-L-lysyl-L- tryptophyl-L-lysyl-L-lysyl-L-seryl-L-aspartylglycylglycyl-O-phosphono-L-tyrosyl-L-methionyl-L-aspartyl-L-methionyl-(740Y-P), 4-(4-cyano-2-fluorophenyl)-2-morpholino-5-(2H1,2,4-triazol-3-yl)thiophene-3-carbonitrile (PF-49892 16), N-(3-{[(3-{[2-chloro-5-(methoxy)phenyl]amino}quinoxalin-2-yl)amino]sulfonyl}phenyl)-2-methylalaninamide (Pilaralisib), 6-((4-(cyclopropylmethyl)piperazin-1-yl)methyl)-2-(5-fluoro-1H-indol-4-yl)-4-morpholinothieno[3,2-d]pyrimidine (PI-3065), 4′-(cyclopropylmethyl)-N2-4-pyridinyl-[4,5′-Bipyrimidine]-2,2′-diamine (PIK-III), 1-[[2-[(2-chloro-4-pyridinyl)amino]-4′-(cyclopropylmethyl)[4,5′-bipyrimidin]-2′-yl]amino]-2-methyl-2-Propanol (VPS34-IN1), 5-(6-(3-methoxyoxetan-3-yl)-7-methyl-4-morpholinothieno[3,2-d]pyrimidin-2-yl)pyrimidin-2-amine (GNE-317), 2-amino-8-ethyl-4-methyl-6-(1H-pyrazol-5-yl)pyrido[2,3-d]pyrimidin-7-one (Voxtalisib), 2-(3,4-dihydroxyphenyl)-3,5,7-trihydroxy-4H-1-Benzopyran-4-one (Quercetin), ethyl 6-(5-(phenylsulfonamido)pyridin-3-yl)imidazo[1,2-a]pyridine-3-carboxylate (HS-173), N-[5-[4-[5-[[(2R,6S)-2,6-dimethyl-4-morpholinyl]methyl]-2-oxazolyl]-1H-indazol-6-yl]-2-methoxy-3-pyridinyl]-methanesulfonamide (GSK2292767), N-((S)-1-(7-fluoro-2-(pyridin-2-yl)quinolin-3-yl)ethyl)-9H-purin-6-amine (AMG3 19), 5-(2-amino-8-fluoro[1,2,4]triazolo[1,5-a]pyridin-6-yl)-N(1,1-dimethylethyl)-3-pyridinesulfonamide (CZC24832), α,α-dimethyl-4-[5,6-dihydro-2-[3-methyl-1-(1-methylethyl)-1H-1,2,4-triazol-5-yl]imidazo[1,2-d][1,4]benzoxazepin-9-yl]-1Hpyrazole-1-acetamide (Taselisib), 5-[8-methyl-9-(1-methylethyl)-2-(4-morpholinyl)-9H-purin-6-yl]-2-pyrimidinamine (VS-5584), 3-(2,4-diamino-6-pteridinyl)-phenol (TG 100713), 1-(4-(3-ethyl7-morpholino-3H-[1,2,3]triazolo[4,5-d]pyrimidin-5-yl)phenyl)-3-(4-(1-methylpiperazine-4-carbonyl)phenyl)urea (PKI-402), 2-((4-amino-1H-pyrazolo[3,4-d]pyrimidin-1-yl)methyl)-5-methyl-3-o-tolylquinazolin-4(3H)-one (PIK-293), (E)-5-((5-(4-fluorophenyl)furan-2-yl)methylene)thiazolidine-2,4-dione (CAY10505), 5-(7-(methylsulfonyl)-2-morpholino-6,7-dihydro-5H-pyrrolo[2,3-d]pyrimidin-4-yl)pyrimidin-2-amine (CH5132799), 6-amino-N-[3-[4-(4-morpholinyl)pyrido[3′,2′:4,5]furo[3,2-d]pyrimidin-2-yl]phenyl]-3-Pyridinecarboxamide (YM201636), 2-amino-N-[2,3-dihydro-7-methoxy-8-[3-(4-morpholinyl)propoxy]imidazo[1,2-c]quinazolin-5-yl]-5-pyrimidinecarboxamide, (Copanlisib), (Z)-5-((2,2-difluorobenzo[d][1,3]dioxol-5-yl)methylene)thiazolidine-2,4-dione (AS-604850), 2-((4-amino-3-(3-hydroxyphenyl)-1H-pyrazolo[3,4-d]pyrimidin-1-yl)methyl)-5-methyl-3-otolylquinazolin-4(3H)-one (PI K-294), N-hydroxy-2-(((2-(6-methoxypyridin-3-yl)-4-morpholinothieno[3,2-d]pyrimidin-6-yl)methyl)(methyl)amino)pyrimidine-5-carboxamide (CUDC-907), 2-methyl-1-[[2-methyl-3-(trifluoromethyl)phenyl]methyl]-6-(4-morpholinyl)-1H-Benzimidazole-4-carboxylic acid (GSK2636771), 8-(6-methoxypyrid in-3-yl)-3-methyl-1-(4-(piperazin-1-yl)-3-(trifluoromethyl)phenyl)-1Himidazo[4,5-c]quinolin-2(3H)-one maleic acid (BGT226), (Z)-5-((5-(4-fluoro-2-hydroxyphenyl)furan-2-yl)methylene)thiazolidine-2,4-dione (AS-252424), 6,7-Bis(3-hydroxyphenyl)pteridine-2,4-diamine (TG100-115), 8-chloro-2-phenyl-3-[(1S)-1-(9H-purin-6-ylamino)ethyl]-1(2H)-isoquinolinone (Duvelisib), 1-(4-(4-(dimethylamino)piperidine-1-carbonyl)phenyl)-3-(4-(4,6-dimorpholino-1,3,5-triazin-2-yl)phenyl)urea (Gedatolisib), (Z)-5-((4-(pyridin-4-yl)quinolin-6-yl)methylene)thiazolidine-2,4-dione (GSK1059615), (S)-1-(4-((2-(2-aminopyrimidin-5-yl)-7-methyl-4-morpholinothieno[3,2-d]pyrimidin-6-yl)methyl)piperazin-1-yl)-2-hydroxypropan-1-one (Apitolisib), 2-amino-8-((1r,4r)-4-(2-hydroxyethoxy)cyclohexyl)-6-(6-methoxypyridin-3-yl)-4-methylpyrido[2,3-d]pyrimidin-7(8H)-one (PF-04691502), (R)-2-(1-(7-methyl-2-morpholino-4-oxo-4H-pyrido[1,2-a]pyrimidin-9-yl)ethylamino)benzoic acid (AZD6482), N-(2,3-dihydro-7,8-dimethoxyimidazo[1,2-c]quinazolin-5-yl)-3-pyridinecarboxamide (PI K-90), 2,4-difluoro-N-(2-methoxy-5-(4-(pyridazin-4-yl)quinolin-6-yl)pyridin-3-yl)benzenesulfonamide (Omipalisib), N-[5-[4-chloro-3-[(2-hydroxyethyl)sulfamoyl]phenyl]-4-methylthiazol-2-yl]acetamide (PIK-93), N-[4-[[[3-[(3,5-dimethoxyphenyl)amino]-2-quinoxalinyl]amino]sulfonyl]phenyl]-3-methoxy-4-methyl-benzamide (Voxtalisib), (2S)-N1-(5-(2-tert-butylthiazol-4-yl)-4-methylthiazol-2-yl)pyrrolidine-1,2-dicarboxamide (A66), (E)-N′-((6-bromoH-imidazo[1,2-a]pyridin-3-yl)methylene)-N,2-dimethyl-5-nitrobenzenesulfonohydrazide hydrochloride (PIK-75 HCl), (Z)-5-(quinoxalin-6-ylmethylene)thiazolidine-2,4-dione (AS-605240), N-1-[4-methyl-5-[2-(2,2,2-trifluoro-1,1-dimethylethyl)-4-pyridinyl]-2-thiazolyl]-(2S)-1,2-pyrrolidinedicarboxamide, (Alpelisib), 2-(difluoromethyl)-1-(4,6-dimorpholino-1,3,5-triazin-2-yl)-1H-benzo[d]imidazole (ZSTK474), 1-[[4′-(cyclopropylmethyl)-2-(4-pyridinylamino) [4,5′-bipyrimidin]-2′-yl]amino]-2-methyl-2-propanol (VPS34 inhibitor 1), 2-[(1S)-1-[4-amino-3-[3-fluoro-4-(1-methylethoxy)phenyl]-1H-pyrazolo[3,4-d]pyrimidin-1-yl]ethyl]-6-fluoro-3-(3-fluorophenyl)-4H-1-benzopyran-4-one (TG R-1202), (8S)-9-[(5-chloro-3-pyridinyl)methyl]-6,7,8,9-tetrahydro-2-[(3R)-3-methyl-4-morpholinyl]-8-(trifluoromethyl)-4H-pyrimido[1,2-a]pyrimidin-4-one (SAR405), (2S)-2-[[5,6-dihydro-2-[1-(1-methylethyl)-1H-1,2,4-triazol-5-yl]imidazo[1,2-d][1,4]benzoxazepin-9-yl]oxy]-propanamide (GDC-0326), 3-(2,3-dihydro-1,4-benzodioxin-6-yl)-5-(4-morpholinyl)-7H-thieno[3,2-b]pyran-7-one (SF2523), 2-amino-N-[(1S)-1-[1,2-dihydro-8-[2-(1-methyl-1H pyrazol-4-yl)ethynyl]-1-oxo-2-phenyl-3-isoquinolinyl]ethyl]-pyrazolo[1,5-a]pyrimidine-3-carboxamide (IPI-549), 5-(2,6-dimorpholinopyrimidin-4-yl)-4-(trifluoromethyl)pyridin-2-amine (Buparlisib), and esters, salts, amines, oxides, hydroxyls, hydroxides, amides, chelates, biosimilars, extensions and truncations thereof.

8. The method of claim 6 wherein said at least one substance is selected from the group consisting of: N4-(7-chloro-4-quinolinyl)-N1,N1-dimethyl-1,4-pentanediamine (chloroquine), and esters, salts, amines, oxides, hydroxyls, hydroxides, amides, chelates, biosimilars, extensions and truncations thereof.

9. The method of claim 6 further comprising delivering at least one anti-oxidant compound selected from the group consisting of: N-acetylcysteine (NAC), α-lipoic acid (ALA), Sadenosylmethionine (SAMe), colecalciferol (vitamin D3), mustard seed and Se rich phyto-supplements.

10. The method of claim 6 further comprising delivering at least one FAAH inhibitor compound.

11. The method of claim 10 wherein said at least one FAAH inhibitor compound is selected from the group consisting of: LEA, OEA and PEA.

12. The method of claim 6 further comprising: delivering at least one anti-inflammatory compound.

13. The method of claim 12 wherein said at least one anti-inflammatory compound is selected from the group consisting of: sulindac, sulindac sulfide, pravadoline, naproxen, naproxen sodium salt, meclofenamate sodium, ibuprophen, S-ibuprofen, piroxicam, ketoprofen, S-ketoprofen, R-ibuprofen, Ebselen, ETYA, diclofenac, diclofenac diethylamine, flurbiprofen, fexofenadine, Pterostilbene, Pterocarpus marsupium, 9,12-octadecadiynoic acid, Ketorolac (tromethamine salt), NO-indomethacin, S-flurbiprofen, sedanolide, green tea extract (e.g., epicatechin), licofelone, lornoxicam, rac ibuprofen-d3, ampirxicam, zaltoprofen, 7-(trifluoromethyl)1H-indole-2,3-dione, aceclofenac, acetylsalicylic acid-d4, S-ibuprofen lysinate, loxoprofen, CAY10589, ZLJ-6, isoicam, dipyrone, YS121 and MEG (mercaptoethylguanidine).

14. The method of claim 6 further comprising delivering at least one compound that activates a receptor selected from the group consisting of: CB1 and CB2.

15. The method of claim 10 wherein said at least one activating compound is selected from the group consisting of: cannabigerolic acid (CBGA) (antibiotic); cannabigerolic acid monomethylether (CBGAM); cannabigerol (CBG) (antibiotic, antifungal, anti-inflammatory, analgesic); Cannabigerol monomethylether (CBGM); cannabigerovarinic acid (CBGVA); Cannabigerovarin (CBGV); Cannabichromenic acid (CBCA); Cannabichromene (CBC) (antibiotic, antifungal, anti-inflammatory, analgesic); Cannabichromevarinic acid (CBCVA); Cannabichromevarin (CBCV); Cannabidiolic acid (CBDA) (antibiotic); Cannabidiol (CBD) ((antioxidant, anxiolytic, antispasmodic, anti-inflammatory, analgesic); cannabidiol monomethylether (CBDM); cannabidiol C4 (CBD-C4); cannabidivarinic acid (CBDVA); cannabidivarin (CBDV); cannabidiorcol (CBD-C1); Δ⁹-tetrahydrocannabinolic acid A (THCA-A); Δ⁹-tetrahydrocannabinolic acid B (THCA-B); 6a, 10a-trans-6a,7,8,10a-tetrahydro-6,6,9-trimethyl-3-pentyl-6H-dibenzo[b,d]pyran-1-ol, (Δ⁹-tetrahydrocannabinol, THC) (analgesic, antioxidant, antiemetic, anti-inflammation); Δ⁹-tetrahydrocannabinolic acid-C4 (THCA-C4); Δ⁹-tetrahydrocan nabinol-C4 (THC-C4); Δ⁹-tetrahydrocannabivarinic acid (THCVA); Δ⁹-tetrahydrocannabivarinic (THCV); Δ⁷-cis-isotetrahydrocannabivarin; Δ⁹-tetrahydrocannabiorcolic acid (THCA-C1); tetrahydrocannabiorcol (THC-C1); Δ⁸-tetrahydrocannabinolic acid (Δ⁸-TCA); Δ⁸-tetrahydrocannabinol (Δ⁸-THC); cannabicyclol (CBL); cannabicyclolicacid (CBLA); cannabicyclovarin (CBLV), cannabiesoic acid A (CBEA-A); cannabiesoic acid B (CBEA-B); cannabieson (CBE); cannabinolic acid (CBNA); cannabinol (CBN); cannabinol methylether (CBNM); cannabinol-C4 (CBN-C4); cannabivarin (CBV); cannabinol-C2 (CBN-C2); cannabiorcol (CBN-C1); cannabinodiol (CBND); cannabinidivarin (CBDV); cannabitriol (CBT); 10-Ethoxy-9-hydroxy-Δ^{6a}-tetrahydrocannabinol (10-EH DT); 8,9-dihydroxy-delta-6a-tetrahydrocannabinol (8,9-DHDT); cannabitriolvarin (CBTV); ethoxy-cannabitriolvarin (CBTVE); dehydrocannabifuran (DCBF); cannabifuran (CBF); cannabichromanon (CBCN); cannabicitran (CBT); 10-oxo-Δ-6a-tetrahydrocannabinol (OTHC); Δ⁹-cis-tetrahydrocannabinol (cis-THC); 3,4,5,6-tetrahydro-7-hydroxy-α-α-2-trimethyl-9-n-propyl-2,6-methano-2H-1-benzoxocin-5-methanol (2H-iso-HHCV); cannabiripsol (CBR); and trihydroxy-Δ⁹-tetrahydrocannabinol (triOHTHC).

16. The method of claim 14 further comprising delivering at least one anti-oxidant compound, at least one FAAH inhibitor compound and at least one anti-inflammatory compound.

17. A pharmaceutical composition comprising at least one substance that inhibits or is metabolized to inhibit performance of the PI3K pathway and at least one antioxidant or antioxidant precursor compound.

18. The pharmaceutical composition of claim 17 wherein said antioxidant or antioxidant precursor acts to maintain GSH level or activity.

19. The pharmaceutical composition of claim 17 wherein said antioxidant or antioxidant precursor compound comprises selenium.

20. The pharmaceutical composition of claim 17 wherein said antioxidant or antioxidant precursor compound is selected from the group consisting of: N-acetylcysteine (NAC), α-lipoic acid (ALA) and S-adenosylmethionine (SAMe).

21. The pharmaceutical composition of claim 17 wherein said at least one substance that inhibits or is metabolized to inhibit performance of the PI3K pathway is selected from the group consisting of: N4-(7-chloro-4-quinolinyl)-N1,N1-dimethyl-1,4-pentanediamine (chloroquine), 2H-dibenzo[cd,g]indazol-6-one (SP600125), 1,4-diamino-2,3-dicyano-1,4-bis(2-aminophenylthio)-butadiene (U0106), Bafilomycin A1 ((3Z,5E,7R,8S,9S,11E,13E,15S,16R)-8-hydroxy-16-[(1S,2R,3S)-2-hydroxy-1-methyl-3-[(2R,4R,5S,6R)-tetrahydro-2,4-dihydroxy-5-methyl-6-(1-methylethyl)-2H-pyran-2-yl]butyl]-3,15-dimethoxy-5,7,9,11-tetramethyloxacyclohexadeca-3,5,11,13-tetraen-2-one (C₃₅H₅₈O₉), (1S,6bR,9aS,11R,11bR)-11-(acetyloxy)-1,6b,7,8,9a,10,11,11b-octahydro-1-(methoxymethyl)-9a, 11b-dimethyl-3H-furo[4,3,2-de]indeno[4,5-h]-2-benzopyran-3,6,9-trione (Wortmann in), 4-(4-(4-fluorophenyl)-2-(4-(methylsulfinyl)phenyl)-1H-imidazol-5-yl)pyridine (SB203580), 4-(4-fluorophenyl)-2-(4-hydroxyphenyl)-5-(4-pyridyl)-1H-imidazole (SB202190), 2-(4-morpholino)-8-phenyl-4H-1-benzopyran-4-one (LY294002), 3-methyladenine (3-MA), 2-methyl-2-{4-[3-methyl-2-oxo-8-(quinolin-3-yl)-2,3-dihydro-1H-imidazo[4,5-c]quinolin-1-yl]phenyl}propanenitrile (Dactolisib), 2-(1H-indazol-4-yl)-6-((4-(methylsulfonyl)piperazin-1-yl)methyl)-4-morpholinothieno[3,2-d]pyrimidine (Pictilisib), (S)-2-(1-(9H-purin-6-ylamino)propyl)-5-fluoro-3-phenylquinazolin4(3H)-one (Idelalisib), (S)-2-(1-(9H-purin-6-ylamino)propyl)-5-fluoro-3-phenylquinazolin-4(3H)-one (AZD8186), (R)-8-(1-(3,5-difluorophenylamino)ethyl)-N,N-dimethyl-2-morpholino-4-oxo-4H-chromene-6-carboxamide (AZD8186), L-serine, L-arginyl-L-glutaminyl-L-isoleucyl-L-lysyl-L-isoleucyl-L-tryptophyl-L-phenylalanyl-L-glutaminyl-L-asparaginyl-L-arginyl-L-arginyl-L-methionyl-L-lysyl-L-tryptophyl-L-lysyl-L-lysyl-L-seryl-L- aspartylglycylglycyl-O-phosphono-L-tyrosyl-L-methionyl-L-aspartyl-L-methionyl-(740Y-P), 4-(4-cyano-2-fluorophenyl)-2-morpholino-5-(2H-1,2,4-triazol-3-yl)thiophene-3-carbonitrile (PF-4989216), N-(3-{[(3-{[2-chloro-5-(methoxy)phenyl]amino}quinoxalin-2-yl)amino]sulfonyl}phenyl)-2-methylalaninamide (Pilaralisib), 6-((4-(cyclopropylmethyl)piperazin-1-yl)methyl)-2-(5-fluoro-1H-indol-4-yl)-4-morpholinothieno[3,2-d]pyrimidine (PI-3065), 4′-(cyclopropylmethyl)-N2-4-pyridinyl-[4,5′-Bipyrimidine]-2,2′-diamine (PIK-III), 1-[[2-[(2-chloro-4-pyridinyl)amino]-4′-(cyclopropylmethyl) [4,5′-bipyrimidin]-2′-yl]amino]-2-methyl-2-propanol (VPS34-IN1), 5-(6-(3-methoxyoxetan-3-yl)-7-methyl-4-morpholinothieno[3,2-d]pyrimidin-2-yl)pyrimidin-2-amine (GNE-317), 2-amino-8-ethyl-4-methyl-6-(1H-pyrazol-5-yl)pyrido[2,3-d]pyrimidin-7-one (Voxtalisib), 2-(3,4-dihydroxyphenyl)-3,5,7-trihydroxy-4H-1-Benzopyran-4-one (Quercetin), ethyl 6-(5-(phenylsulfonamido)pyridin-3-yl)imidazo[1,2-a]pyridine-3-carboxylate (HS-173), N-[5-[4-[5-[[(2R,6S)-2,6-dimethyl-4-morpholinyl]methyl]-2-oxazolyl]-1H-indazol-6-yl]-2-methoxy-3-pyridinyl]-methanesulfonamide (GSK2292767), N-((S)-1-(7-fluoro-2-(pyridin-2-yl)quinolin-3-yl)ethyl)-9H-purin-6-amine (AMG3 19), 5-(2-amino-8-fluoro[1,2,4]triazolo[1,5-a]pyridin-6-yl)-N(1,1-dimethylethyl)-3-pyridinesulfonamide (CZC24832), α,α-dimethyl-4-[5,6-dihydro-2-[3-methyl-1-(1-methylethyl)-1H-1,2,4-triazol-5-yl]imidazo[1,2-d][1,4]benzoxazepin-9-yl]-1H-pyrazole-1-acetamide (Taselisib), 5-[8-methyl-9-(1-methylethyl)-2-(4-morpholinyl)-9H-purin-6-yl]-2-pyrimidinamine (VS-5584), 3-(2,4-diamino-6-pteridinyl)-phenol (TG 100713), 1-(4-(3-ethyl7-morpholino-3H-[1,2,3]triazolo[4,5-d]pyrimidin-5-yl)phenyl)-3-(4-(1-methylpiperazine-4-carbonyl)phenyl)urea (PKI-402), 2-((4-amino-1H-pyrazolo[3,4-d]pyrimidin-1-yl)methyl)-5-methyl-3-o-tolylquinazolin-4(3H)-one (PIK-293), (E)-5-((5-(4-fluorophenyl)furan-2-yl)methylene)thiazolidine-2,4-dione (CAY10505), 5-(7-(methylsulfonyl)-2-morpholino-6,7-dihydro-5H-pyrrolo[2,3-d]pyrimidin-4-yl)pyrimidin-2-amine (CH5132799), 6-amino-N-[3-[4-(4-morpholinyl)pyrido[3′,2′:4,5]furo[3,2-d]pyrimidin-2-yl]phenyl]-3-pyridinecarboxamide (YM201636), 2-amino-N-[2,3-dihydro-7-methoxy-8-[3-(4-morpholinyl)propoxy]imidazo[1,2-c]quinazolin-5-yl]-5-pyrimidinecarboxamide, (Copanlisib), (Z)-5-((2,2-difluorobenzo[d][1,3]dioxol-5-yl)methylene)thiazolidine-2,4-dione (AS-604850), 2-((4-amino-3-(3-hydroxyphenyl)-1H-pyrazolo[3,4-d]pyrimidin-1-yl)methyl)-5-methyl-3-otolylquinazolin-4(3H)-one (PI K-294), N-hydroxy-2-(((2-(6-methoxypyridin-3-yl)-4-morpholinothieno[3,2-d]pyrimidin-6-yl)methyl)(methyl)amino)pyrimidine-5-carboxamide (CUDC-907), 2-methyl-1-[[2-methyl-3-(trifluoromethyl)phenyl]methyl]-6-(4-morpholinyl)-1H-Benzimidazole-4-carboxylic acid (GSK2636771), 8-(6-methoxypyridin-3-yl)-3-methyl-1-(4-(piperazin-1-yl)-3-(trifluoromethyl)phenyl)-1Himidazo[4,5-c]quinolin-2(3H)-one maleic acid (BGT226), (Z)-5-((5-(4-fluoro-2-hydroxyphenyl)furan-2-yl)methylene)thiazolidine-2,4-dione (AS-252424), 6,7-bis(3-hydroxyphenyl)pteridine-2,4-diamine (TG100-115), 8-chloro-2-phenyl-3-[(1S)-1-(9H-purin-6-ylamino)ethyl]-1(2H)-Isoquinolinone (Duvelisib), 1-(4-(4-(dimethylamino)piperidine-1-carbonyl)phenyl)-3-(4-(4,6-dimorpholino-1,3,5-triazin-2-yl)phenyl)urea (Gedatolisib), (Z)-5-((4-(pyridin-4-yl)quinolin-6-yl)methylene)thiazolidine-2,4-dione (GSK1059615), (S)-1-(4-((2-(2-aminopyrimidin-5-yl)-7-methyl-4-morpholinothieno[3,2-d]pyrimidin-6-yl)methyl)piperazin-1-yl)-2-hydroxypropan-1-one (Apitolisib), 2-amino-8-((1r,4r)-4-(2-hydroxyethoxy)cyclohexyl)-6-(6-methoxypyridin-3-yl)-4-methylpyrido[2,3-d]pyrimidin-7(8H)-one (PF-04691502), (R)-2-(1-(7-methyl-2-morpholino-4-oxo-4H-pyrido[1,2-a]pyrimidin-9-yl)ethylamino)benzoic acid (AZD6482), N-(2,3-dihydro-7,8-dimethoxyimidazo[1,2-c]quinazolin-5-yl)-3-pyridinecarboxamide (PI K-90), 2,4-difluoro-N-(2-methoxy-5-(4-(pyridazin-4-yl)quinolin-6-yl)pyridin-3-yl)benzenesulfonamide (Omipalisib), N-[5-[4-chloro-3-[(2-hydroxyethyl)sulfamoyl]phenyl]-4-methylthiazol-2-yl]acetamide (PIK-93), N-[4-[[[3-[(3,5-dimethoxyphenyl)amino]-2-quinoxalinyl]amino]sulfonyl]phenyl]-3-methoxy-4-methyl-benzamide (Voxtalisib), (2S)-N1-(5-(2-tert-butylthiazol-4-yl)-4-methylthiazol-2-yl)pyrrolidine-1,2-dicarboxamide (A66), (E)-N′-((6-bromoH-imidazo[1,2-a]pyridin-3-yl)methylene)-N,2-dimethyl-5-nitrobenzenesulfonohydrazide hydrochloride (PIK-75 HCl), (Z)-5-(quinoxalin-6-ylmethylene)thiazolidine-2,4-dione (AS-605240), N-1-[4-methyl-5-[2-(2,2,2-trifluoro-1,1-dimethylethyl)-4-pyridinyl]-2-thiazolyl]-(2S)-1,2-pyrrolidinedicarboxamide, (Alpelisib), 2-(difluoromethyl)-1-(4,6-dimorpholino-1,3,5-triazin-2-yl)-1H-benzo[d]imidazole (ZSTK474), 1-[[4′-(cyclopropylmethyl)-2-(4-pyridinylamino)[4,5′-bipyrimidin]-2′-yl]amino]-2-methyl-2-propanol (VPS34 inhibitor 1), 2-[(1S)-1-[4-amino-3-[3-fluoro-4-(1-methylethoxy)phenyl]-1H-pyrazol o[3,4-d]pyrimidin-1-yl]ethyl]-6-fluoro-3-(3-fluorophenyl)-4H-1-Benzopyran-4-one (TG R-1202), (8S)-9-[(5-chloro-3-pyridinyl)methyl]-6,7,8,9-tetrahydro-2-[(3R)-3-methyl-4-morpholinyl]-8-(trifluoromethyl)-4H-pyrimido[1,2-a]pyrimidin-4-one (SAR405), (2S)-2-[[5,6-dihydro-2-[1-(1-methylethyl)-1H-1,2,4-triazol-5-yl]imidazo[1,2-d][1,4]benzoxazepin-9-yl]oxy]-propanamide (GDC-0326), 3-(2,3-dihydro-1,4-benzodioxin-6-yl)-5-(4-morpholinyl)-7H-thieno[3,2-b]pyran-7-one (SF2523), 2-amino-N-[(1S)-1-[1,2-dihydro-8-[2-(1-methyl-1H pyrazol-4-yl)ethynyl]-1-oxo-2-phenyl-3-isoquinolinyl]ethyl]-pyrazolo[1,5-a]pyrimidine-3-carboxamide (IPI-549), 5-(2,6-dimorpholinopyrimidin-4-yl)-4-(trifluoromethyl)pyridin-2-amine (Buparlisib), and esters, salts, amines, oxides, hydroxyls, hydroxides, amides, chelates, biosimilars, extensions and truncations thereof.

22. The pharmaceutical composition of claim 17 further comprising at least one divalent cation, salt of divalent cation, or divalent cation chelate.

23. The pharmaceutical composition of claim 22 wherein said divalent cation or chelated divalent cation is selected from the group consisting of: Ca⁺⁺ and Mg⁺⁺.

24. The pharmaceutical composition of claim 17 comprising colecalciferol (vitamin D3).

25. The pharmaceutical composition of claim 17 further comprising: at least one compound selected from the group consisting of: a compound that activates a receptor selected from the group consisting of: CB1 and CB2; and at least one FAAH inhibitor compound selected from the group consisting of: LEA, OEA and PEA.

26. A medicament formulated with an inhibitor of PI3K for arresting an overzealous immune activity in a person or animal.

27. A compound selected from the group consisting of: N4-(7-Chloro-4-quinolinyl)-N1,N1-dimethyl-1,4-pentanediamine (chloroquine), 2H-dibenzo[cd,g]indazol-6-one (SP600125), 1,4-Diamino-2,3-dicyano-1,4-bis(2-aminophenylthio)-butadiene (U0106), Bafilomycin A1 ((3Z,5E,7R,8S,9S,11E,13E,15S,16R)-8-Hydroxy-16-[(1S,2R,3S)-2-hydroxy-1-methyl-3-[(2R,4R,5S,6R)-tetrahydro-2,4-dihydroxy-5-methyl-6-(1-methylethyl)-2H-pyran-2-yl]butyl]-3,15-dimethoxy-5,7,9,11-tetramethyloxacyclohexadeca-3,5,11,13-tetraen-2-one (C35H58O9), (1S,6bR,9aS,11R,11bR)-11-(acetyloxy)-1,6b,7,8,9a,10,11,11b-octahydro-1-(methoxymethyl)-9a,11b-dimethyl-3H-Furo[4,3,2-de]indeno[4,5-h]-2-benzopyran-3,6,9-trione (Wortmannin), 4-(4-(4-fluorophenyl)-2-(4-(methylsulfinyl)phenyl)-1H-imidazol-5-yl)pyridine (SB203580), 4-(4-Fluorophenyl)-2-(4-hydroxyphenyl)-5-(4-pyridyl)-1H-imidazole (SB202190), 2-(4-morpholino)-8-phenyl-4H-1-benzopyran-4-one (LY294002), 3-methyladenine (3-MA), 2-Methyl-2-{4-[3-methyl2-oxo-8-(quinolin-3-yl)-2,3-dihydro-1H-imidazo[4,5-c]quinolin-1-yl]phenyl}propanenitrile (Dactolisib), 2-(1H-indazol-4-yl)-6-((4-(methylsulfonyl)piperazin-1-yl)methyl)-4-morpholinothieno[3,2-d]pyrimidine (Pictilisib), (S)-2-(1-(9H-purin-6-ylamino)propyl)-5-fluoro-3-phenylquinazolin-4(3H)-one (Idelalisib), (S)-2-(1-(9H-purin-6-ylamino)propyl)-5-fluoro-3-phenylquinazolin-4(3H)-one (AZD8186), (R)-8-(1-(3,5-difluorophenylamino)ethyl)-N,N-dimethyl2-morpholino-4-oxo-4H-chromene-6-carboxamide (AZD8186), L-Serine, L-arginyl-L-glutaminyl-L-isoleucyl-L-lysyl-L-isoleucyl-L-tryptophyl-L-phenylalanyl-L-glutaminyl-L-asparaginyl-L-arginyl-L-arginyl-L-methionyl-L-lysyl-L-tryptophyl-L-lysyl-L-lysyl-L-seryl-L- aspartylglycylglycyl-O-phosphono-L-tyrosyl-L-methionyl-L-aspartyl-L-methionyl-(740Y-P), 4-(4-cyano-2-fluorophenyl)-2-morpholino-5-(2H-1,2,4-triazol-3-yl)thiophene-3-carbonitrile (PF-4989216), N-(3-{[(3-{[2-chloro-5-(methoxy)phenyl]amino}quinoxalin-2-yl)amino]sulfonyl}phenyl)-2-methylalaninamide (Pilaralisib), 6-((4-(cyclopropylmethyl)piperazin-1-yl)methyl)-2-(5-fluoro-1H-indol-4-yl)-4-morpholinothieno[3,2-d]pyrimidine (PI-3065), 4′-(cyclopropylmethyl)-N2-4-pyridinyl-[4,5′-Bipyrimidine]-2,2′-diamine (PIK-III), 1-[[2-[(2-chloro-4-pyridinyl)amino]-4′-(cyclopropylmethyl)[4,5′-bipyrimidin]-2′-yl]amino]-2-methyl-2-Propanol (VPS34-IN1), 5-(6-(3-methoxyoxetan-3-yl)-7-methyl-4-morpholinothieno[3,2-d]pyrimidin-2-yl)pyrimidin-2-amine (GNE-317), 2-amino-8-ethyl-4-methyl-6-(1H-pyrazol-5-yl)pyrido[2,3-d]pyrimidin-7-one (Voxtalisib), 2-(3,4-dihydroxyphenyl)-3,5,7-trihydroxy-4H-1-Benzopyran-4-one (Quercetin), Ethyl 6-(5-(phenylsulfonamido)pyridin-3-yl)imidazo[1,2-a]pyridine-3-carboxylate (HS-173), N-[5-[4-[5-[[(2R,6S)-2,6-dimethyl-4-morpholinyl]methyl]-2-oxazolyl]-1H-indazol-6-yl]-2-methoxy-3-pyridinyl]-methanesulfonamide (GSK2292767), N-((S)-1-(7-fluoro-2-(pyridin-2-yl)quinolin-3-yl)ethyl)-9H-purin-6-amine (AMG3 19), 5-(2-amino-8-fluoro[1,2,4]triazolo[1,5-a]pyridin-6-yl)-N(1,1-dimethylethyl)-3-Pyridinesulfonamide (CZC24832), α,α-dimethyl-4-[5,6-dihydro-2-[3-methyl-1-(1-methylethyl)-1H-1,2,4-triazol-5-yl]imidazo[1,2-d][1,4]benzoxazepin-9-yl]-1Hpyrazole-1-acetamide (Taselisib), 5-[8-methyl-9-(1-methylethyl)-2-(4-morpholinyl)-9H-purin-6-yl]-2-pyrimidinamine (VS-5584), 3-(2,4-diamino-6-pteridinyl)-phenol (TG 100713), 1-(4-(3-ethyl7-morpholino-3H-[1,2,3]triazolo[4,5-d]pyrimidin-5-yl)phenyl)-3-(4-(1-methylpiperazine-4-carbonyl)phenyl)urea (PKI-402), 2-((4-amino-1H-pyrazolo[3,4-d]pyrimidin-1-yl)methyl)-5-methyl-3-o-tolylquinazolin-4(3H)-one (PIK-293), (E)-5-((5-(4-fluorophenyl)furan-2-yl)methylene)thiazolidine-2,4-dione (CAY10505), 5-(7-(methylsulfonyl)-2-morpholino-6,7-dihydro-5H-pyrrolo[2,3-d]pyrimidin-4-yl)pyrimidin-2-amine (CH5132799), 6-amino-N-[3-[4-(4-morpholinyl)pyrido[3′,2′:4,5]furo[3,2-d]pyrimidin-2-yl]phenyl]-3-Pyridinecarboxamide (YM201636), 2-amino-N-[2,3-dihydro-7-methoxy-8-[3-(4-morpholinyl)propoxy]imidazo[1,2-c]quinazolin-5-yl]-5-pyrimidinecarboxamide, (Copanlisib), (Z)-5-((2,2-difluorobenzo[d][1,3]dioxol-5-yl)methylene)thiazolidine-2,4-dione (AS-604850), 2-((4-amino-3-(3-hydroxyphenyl)-1H-pyrazolo[3,4-d]pyrimidin-1-yl)methyl)-5-methyl-3-otolylquinazolin-4(3H)-one (PI K-294), N-hydroxy-2-(((2-(6-methoxypyridin-3-yl)-4-morpholinothieno[3,2-d]pyrimidin-6-yl)methyl)(methyl)amino)pyrimidine-5-carboxamide (CUDC-907), 2-methyl-1-[[2-methyl-3-(trifluoromethyl)phenyl]methyl]-6-(4-morpholinyl)-1H-Benzimidazole-4-carboxylic acid (GSK2636771), 8-(6-methoxypyrid in-3-yl)-3-methyl-1-(4-(piperazin-1-yl)-3-(trifluoromethyl)phenyl)-1Himidazo[4,5-c]quinolin-2(3H)-one maleic acid (BGT226), (Z)-5-((5-(4-fluoro-2-hydroxyphenyl)furan-2-yl)methylene)thiazolidine-2,4-dione (AS-252424), 6,7-Bis(3-hydroxyphenyl)pteridine-2,4-diamine (TG100-115), 8-chloro-2-phenyl-3-[(1S)-1-(9H-purin-6-ylamino)ethyl]-1(2H)-Isoquinolinone (Duvelisib), 1-(4-(4-(dimethylamino)piperidine-1-carbonyl)phenyl)-3-(4-(4,6-dimorpholino-1,3,5-triazin-2-yl)phenyl)urea (Gedatolisib), (Z)-5-((4-(pyridin-4-yl)quinolin-6-yl)methylene)thiazolidine-2,4-dione (GSK1059615), (S)-1-(4-((2-(2-aminopyrimidin-5-yl)-7-methyl-4-morpholinothieno[3,2-d]pyrimidin-6-yl)methyl)piperazin-1-yl)-2-hydroxypropan-1-one (Apitolisib), 2-amino-8-((1r,4r)-4-(2-hydroxyethoxy)cyclohexyl)-6-(6-methoxypyridin-3-yl)-4-methylpyrido[2,3-d]pyrimidin-7(8H)-one (PF-0469 1502), (R)-2-(1-(7-methyl-2-morpholino-4-oxo-4H-pyrido[1,2-a]pyrimidin-9-yl)ethylamino)benzoic acid (AZD6482), N-(2,3-dihydro-7,8-dimethoxyimidazo[1,2-c]quinazolin-5-yl)-3-pyridinecarboxamide (PI K-90), 2,4-difluoro-N-(2-methoxy-5-(4-(pyridazin-4-yl)quinolin-6-yl)pyridin-3-yl)benzenesulfonamide (Omipalisib), N-[5-[4-Chloro-3-[(2-hydroxyethyl)sulfamoyl]phenyl]-4-methylthiazol-2-yl]acetamide (PIK-93), N-[4-[[[3-[(3,5-dimethoxyphenyl)amino]-2-quinoxalinyl]amino]sulfonyl]phenyl]-3-methoxy-4-methyl-benzamide (Voxtalisib), (2S)-N1-(5-(2-tert-butylthiazol-4-yl)-4-methylthiazol-2-yl)pyrrolidine-1,2-dicarboxamide (A66), (E)-N′-((6-bromoH-imidazo[1,2-a]pyridin-3-yl)methylene)-N,2-dimethyl-5-nitrobenzenesulfonohydrazide hydrochloride (PIK-75 HCl), (Z)-5-(quinoxalin-6-ylmethylene)thiazolidine-2,4-dione (AS-605240), N-1-[4-methyl-5-[2-(2,2,2-trifluoro-1,1-dimethylethyl)-4-pyridinyl]-2-thiazolyl]-(2S)-1,2-pyrrolidinedicarboxamide, (Alpelisib), 2-(difluoromethyl)-1-(4,6-dimorpholino-1,3,5-triazin-2-yl)-1H-benzo[d]imidazole (ZSTK474), 1-[[4′-(cyclopropylmethyl)-2-(4-pyridinylamino)[4,5′-bipyrimidin]-2′-yl]amino]-2-methyl-2-propanol (VPS34 inhibitor 1), 2-[(1S)-1-[4-amino-3-[3-fluoro-4-(1-methylethoxy)phenyl]-1H-pyrazolo[[3,4-d]pyrimidin-1-yl]ethyl]-6-fluoro-3-(3-fluorophenyl)-4H-1-Benzopyran-4-one (TGR-1202), (8S)-9-[(5-chloro-3-pyridinyl)methyl]-6,7,8,9-tetrahydro-2-[(3R)-3-methyl-4-morpholinyl]-8-(trifluoromethyl)-4H-pyrimido[1,2-a]pyrimidin-4-one (SAR405), (2S)-2-[[5,6-dihydro-2-[1-(1-methylethyl)-1H-1,2,4-triazol-5-yl]imidazo[1,2-d][1,4]benzoxazepin-9-yl]oxy]-propanamide (GDC-0326), 3-(2,3-dihydro-1,4-benzodioxin-6-yl)-5-(4-morpholinyl)-7H-thieno[3,2-b]pyran-7-one (SF2523), 2-amino-N-[(1S)-1-[1,2-dihydro-8-[2-(1-methyl-1H pyrazol-4-yl)ethynyl]-1-oxo-2-phenyl-3-isoquinolinyl]ethyl]-pyrazolo[1,5-a]pyrimidine-3-carboxamide (IPI-549), 5-(2,6-dimorpholinopyrimidin-4-yl)-4-(trifluoromethyl)pyridin-2-amine (Buparlisib), and esters, salts, amines, oxides, hydroxyls, hydroxides, amides, chelates, biosimilars, extensions and truncations thereof for treating an autoimmune disease.
